(19) United States
(12) Patent Application Publication
(10) Pub. No.: US 2013/0331320 A1
(43) Pub. Date: Dec. 12, 2013

(54) FAST-ACTING INSULIN IN COMBINATION WITH LONG-ACTING INSULIN

(71) Applicants: Svend Havelund, Bagsvaerd (DK); Ulla Ribel-Madsen, Virum (DK); Ib Jonassen, Jaegerspris (DK); Helle Birk Olsen, Alleroed (DK)

(72) Inventors: Svend Havelund, Bagsvaerd (DK); Ulla Ribel-Madsen, Virum (DK); Ib Jonassen, Jaegerspris (DK); Helle Birk Olsen, Alleroed (DK)

(73) Assignee: Novo Nordisk A/S, Bagsvaerd (DK)

(21) Appl. No.: 13/993,214

(22) PCT Filed: Dec. 14, 2011
(86) PCT No.: PCT/EP11/72739

§ 371 Date: Aug. 27, 2013

Related U.S. Application Data
(60) Provisional application No. 61/425,370, filed on Dec. 21, 2010.

(30) Foreign Application Priority Data
Dec. 14, 2010 (EP) 10194902.2

(51) Int. Cl. A61K 38/28

(52) U.S. Cl. CPC A61K 38/28
USPC 514/6.2; 514/6.3; 514/6.5

(57) ABSTRACT

Insulin preparations comprising a long-acting insulin compound, a fast-acting insulin compound, a nicotinic compound and an amino acid.

### FIELD OF THE INVENTION

The present invention relates to insulin preparations comprising a long-acting insulin compound, a fast-acting insulin compound, a nicotinic compound and an amino acid. The present invention also relates to a method for producing an insulin preparation with both a prolonged action profile and a rapid action profile and a method for manufacturing a pharmaceutical composition for the treatment of diabetes.

### BACKGROUND OF THE INVENTION

Diabetes mellitus is a metabolic disorder in which the ability to utilize glucose is partly or completely lost. About 5% of all people suffer from diabetes and the disorder approaches epidemic proportions.

Since the introduction of insulin in the 1920's, continuous improvements have been made in the treatment of diabetes. To help avoid high glycaemia levels, diabetic patients often practice multiple injection therapy, whereby insulin is administered with each meal. As diabetic patients have been treated with insulin for several decades, there is a major need for safe and life-quality improving insulin preparations. Among the commercially available insulin preparations, rapid-acting, intermediate-acting and prolonged-acting preparations can be mentioned.

Currently, the treatment of diabetes, both type 1 diabetes and type 2 diabetes, relies to an increasing extent on the so-called intensive insulin treatment. According to this regimen, the patients are treated with multiple daily insulin injections comprising one or two daily injections of a long acting insulin to cover the basal insulin requirement supplemented by bolus injections of a rapid acting insulin to cover the insulin requirement related to meals.

In the treatment of diabetes mellitus, many varieties of pharmaceutical preparations of insulin have been suggested and used, such as regular insulin (such as Actrapid®), isophane insulin (designated NPH), insulin zinc suspensions (such as Semilente®, Lente®, and Ultralente®), and biphasic isophane insulin (such as NovoMix®). Human insulin analogues and derivatives have also been developed, designed for particular profiles of action, i.e. fast action or prolonged action. The long-acting insulin analogue, degludec; is currently in fase 3a clinic (Begin™), as well as a biphasic preparation of degludec and the fast-acting insulin aspart, DegludecPlus, has entered phase 3 clinic (BOOST™). Some of the commercially available insulin preparations comprising rapid acting insulin analogues include NovoRapid® (preparation of B28Asp human insulin), Humalog® (preparation of B28LysB29Pro human insulin) and Apidra® (preparation of B3LysB29Glu human insulin). Some of the commercially available insulin preparations comprising long-acting insulin analogues include Lantus® (preparation of insulin glargine) and Levemir® (preparation of insulin detemir).

International applications WO 91/09617 and WO/9610417 (Novo Nordisk A/S) disclose insulin preparations containing nicotinamide or nicotinic acid or a salt thereof.

Most often pharmaceutical preparations of insulins are administered by subcutaneous injection. Important for the patient is the action profile of the insulin, meaning the action of insulin on glucose metabolism as a function of time from injection. In this profile, inter alia, the time of the onset, the maximum value and the total duration of action are important. In the case of bolus insulins, a variety of insulin preparations with different action profiles are desired and requested by the patients. One patient may, on the same day, use insulin preparations with very different action profiles. The action profile desired for example, depends on the time of the day and the amount and composition of the meal eaten by the patient.

A distinctive property of insulin is its ability to associate into hexamers, in which form the hormone is protected from chemical and physical degradation during biosynthesis and storage. X-ray crystallographic studies on insulin show that the hexamer consists of three dimers related by a 3-fold axis of rotation. These dimers are closely associated through the interaction of two zinc ions at its core positioned on the 3-fold axis. When human insulin is injected into the subcutis in the form of a high-concentration pharmaceutical formulation it is self associated, and here dissociation into monomers is relatively slow. Hexamers and dimers of insulin are slower to penetrate capillary wall than monomers.

WO 2003/094956 and WO 2003/094951 disclose stable insulin having both fast and long action (acylated insulin, insulin detemir). WO 2007/074133 discloses a composition comprising an long-acting acylated insulin (degludec) and a rapid acting insulin (insulin aspart).

Equally important for the patient is the chemical stability of the insulin preparations, for example, due to the abundant use of pen-like injection devices such as devices which contain Penfill® cartridges, in which an insulin preparation is stored until the entire cartridge is empty which may be at least 1 to 2 weeks for devices containing 1.5-3.0 ml cartridges. During storage, covalent chemical changes in the insulin structure occur. This may lead to formation of molecules which may be less active and/or potentially immunogenic such as deamidation products and higher molecular weight transformation products (dimers, polymers). Furthermore, also important is the physical stability of the insulin preparations, since long term storage may eventually lead to formation of insoluble fibrils, which are biologically inactive and potentially immunogenic.

### SUMMARY OF THE INVENTION

The present invention relates to insulin preparations comprising a long-acting insulin compound, a fast-acting insulin compound, a nicotinic compound and/or salts thereof and an amino acid.

The invention relates to insulin preparations with improved absorption rate of the fast-acting insulin compound, while maintaining a prolonged action profile of the long-acting insulin compound. The present invention further relates to preparations with favourable chemical and physical stability.

In one embodiment, the present invention relates to an insulin preparation comprising:
a long-acting insulin compound, which is an acylated insulin or acylated insulin analog
a fast-acting insulin compound, which is an insulin analog or human insulin,
a nicotinic compound, and
arginine

In another embodiment, the present invention also contemplates a method for the treatment of diabetes mellitus in a subject or for reducing the blood glucose level in a subject comprising administering to a subject or mammal an insulin preparation according to the invention.

### DESCRIPTION OF THE DRAWINGS

FIG. 1 shows the absorption rate of insulin aspart in a Boost™ formulation (grey line) is increased by including 80 mM (dotted line), 120 mM (full line), and 230 mM (dashed line) nicotinamide in the preparations (Example 3).

FIG. 2 shows the kinetic profile of insulin degludec in a Boost™ formulation (gray line) is changed by including 230 mM nicotinamide (dashed line), whereas formulations including 80 mM (dotted line) or 120 mM (full line) nicotinamide are similar to the reference (Example 3).

FIG. 3 shows multihexamer formation of insulin degludec in preparations combined with insulin aspart according to Table 1 was reduced by including 230 mM (dashed line) or 120 mM nicotinamide (solid line) whereas the peak height of the multihexameric complex was about the same for preparations including 80 mM (dotted line) or 40 mM nicotinamide (dash dot line) as a reference preparation without nicotinamide (grey solid) according to an in vitro model using size exclusion chromatography on a Superose 6PC column in buffered saline. (Example 5).

### DESCRIPTION OF THE INVENTION

The present invention relates to insulin preparations comprising a long-acting insulin compound, a fast-acting insulin compound, a nicotinic compound and/or salts thereof and an amino acid.

The absorption after subcutaneous injection of the fast-acting insulin compound in the insulin preparations of the present invention was surprisingly found to be faster than that of the reference insulin preparations. This property is useful for rapid-acting insulins, in particular in connection with a multiple injection regimen where insulin is given before each meal. With faster onset of action, the insulin can conveniently be taken closer to the meal than with conventional rapid acting insulin solutions. Furthermore, a faster disappearance of insulin probably diminishes the risk of post-meal hypoglycaemia. At the same time, the formation of multihexamers of the long-acting insulin compound in the insulin preparations of the present invention, remained favourable for the long-acting insulin compound.

The insulin preparations of the present invention are mix preparations comprising a long-acting insulin compound, such as insulin degludec, a rapid-acting insulin compound such as insulin aspart, a nicotinic compound, such as nicotinamide and the amino acid arginine. In one embodiment, the insulin preparations of the present invention may comprise other amino acids. These insulin preparations have a combined rapid absorption and ultra-long profiles that mimics normal physiology more closely than existing therapies. Furthermore, the insulin preparations of the present invention have chemical and physical stability suitable for commercial pharmaceutical preparations.

The insulin preparations of the present invention provide an even faster onset of action of the fast-acting insulin compound and without altering the ultra long-acting profile of the long-acting insulin compound compared with existing insulin therapies. The ultra-fast insulin compound in the preparations have the advantage of restoring first phase insulin release, injection convenience and shutting down hepatic glucose production. The insulin preparations of the present invention have a favourable absorption rate from subcutis into plasma with an increase in initial absorption rate ranging from 1.5 to 3 times, when compared to conventional preparations such as BOOST™, as suggested by several PK/PD experiments in pigs. This faster absorption rate may improve glycaemic control and convenience and may allow for a shift from pre-meal to post-meal dosing. The present invention is based in part, on the surprising discovery that although, the addition of nicotinamide allows the increase in absorption rate of the rapid acting insulin analogue, it also has a negative effect on chemical stability by significantly increasing the amount of HMWP. The insulin preparations of the present invention have an improved chemical stability by addition of arginine, which is reflected in e.g. a reduction in the formation of dimers and polymers and desamido insulins after storage.

Addition of high concentrations of nicotinamide was shown to alter the long-acting profile of degludec in a pig model, whereas lower concentrations of nicotinamide had no impact on degludec profile while still increasing the absorption rate of insulin aspart. Similarly, at higher concentrations of nicotinamide there was reduced degludec multi-hexamer formation in the composition and low impact on multihexamer formation at lower concentrations of nicotinamide in the composition.

In one embodiment of the present invention, the nicotinic compound is present in the composition at a concentration less than 260 mM or less than 230 mM.

In one embodiment the insulin preparations comprise a long-acting insulin compound, a fast-acting insulin compound or combinations thereof, a nicotinic compound and/or salts thereof and arginine and/or salts thereof.

The present invention provides insulin preparations comprising a fast-acting insulin compound and a long-acting insulin compound according to the present invention, which are present in a concentration from about 0.1 mM to about 10.0 mM, and wherein said preparation has a pH from 3 to 8.5. The preparations also comprise a nicotinic compound and arginine. The preparations may further comprise metal ions, a buffer system, preservative(s), tonicity agent(s), chelating agent(s), stabilizers and/or surfactants.

In one embodiment, the long-acting insulin is an acylated insulin analogue.

In another embodiment, the acylated insulin analogue is Nε1329-hexadecandiyol-γ-Glu-(desB30) human insulin.

In one embodiment, the insulin preparations according to the present invention comprise an aqueous solution of Nε1329-hexadecandiyol-γ-Glu-(desB30) human insulin, B28Asp human insulin, nicotinamide and arginine. The content of Nε1329-hexadecandiyol-γ-Glu-(desB30) human insulin in the preparations of this invention may be in the range of 15 to 500 international units (IU)/ml, for example in the range of 30 to 333 IU/ml, in preparations for injection. The content of B28Asp human insulin in the solutions of this invention may be in the range of 15 to 500 international units (IU)/ml, for example in the range of 30 to 333 IU/ml, in preparations for injection. However, for other purposes of parenteral administration, the content of insulin compound may be higher.

In the present context the unit “IU” corresponds to 6 nmol.

The term “insulin degludec” or “degludec” refers to the acylated human insulin analogue NεB29-hexadecandiyol-γ-Glu-(desB30) human insulin.

The term “insulin aspart” or “aspart” refers to the human insulin analogue B28Asp human insulin.

The term “onset” refers to the time from injection until the PK curve shifts to an increase.

The term “absorption rate” refers to the slope of the PK curve.

An “insulin compound” according to the invention is herein to be understood as human insulin, an insulin analogue and/or any combination thereof.

The term “human insulin” as used herein means the human hormone whose structure and properties are well-known. Human insulin has two polypeptide chains that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain, the second between the cysteine in position 7 of the A-chain and the cysteine in position 7 of the B-chain, and the third between the cysteine in position 20 of the A-chain and the cysteine in position 19 of the B-chain.

The hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

The term “basal insulin” as used herein means an formulation of insulin peptide which has a time-action of more than 15 hours in standard models of diabetes and is suited to cover the need for insulin during the night and in-between meals. Preferably, the basal insulin has a time-action of at least 20 hours. Preferably, the basal insulin has a time-action of at least 10 hours. Preferably, the basal insulin has a time-action in the range from 15 to 48 hours. Preferably, the basal insulin has a time-action similar to or longer than that observed for commercial pharmaceutical compositions of NPH insulin or N^{εB29}-tetradecanoyl desB30 human insulin.

The term “bolus insulin”, “meal-related insulin” or “rapid acting insulin” as used herein means an insulin peptide which is rapid-acting and suited to cover the need for insulin during and after the meal.

The term “biphasic insulin” as used herein means a pharmaceutical composition comprising a mixture of “bolus insulin” and “basal insulin”.

The term “no blunting” as used herein means that when formulated in one formulation both the rapid acting insulin and the acylated insulin has profile of action which is identical or substantially identical with the profile of action, when administering the rapid acting insulin and the acylated insulin in separate formulations.

The term “OAD” or “OAD(s)” as used herein means oral antidiabetic drug or oral antidiabetic drugs. An unlimited list of OAD(s) can be sulfonylurea (SU), biguanides e.g. Melformin or thiozolidindiones (TZD).

The expression “a codable amino acid” or “a codable amino acid residue” is used to indicate an amino acid or amino acid residue which can be coded for by a triplet (“codon”) of nucleotides.

hGlu is homoglutamic acid.

α-Asp is the L-form of —HNCH(CO—)CH₂COOH.

β-Asp is the L-form of —HNCH(COOH)CH₂CO—.

α-Glu is the L-form of —HNCH(CO—)CH₂CH₂COOH.

γ-Glu is the L-form of —HNCH(COOH)CH₂CH₂CO—.

α-hGlu is the L-form of —HNCH(CO—)CH₂CH₂CH₂COOH.

δ-hGlu is the L-form of —HNCH(COOH)CH₂CH₂CH₂CO—.

β-Ala is —NH—CH₂—CH₂—COOH.

Sar is sarcosine (N-methylglycine).

The expression “an amino acid residue having a carboxylic acid group in the side chain” designates amino acid residues like Asp, Glu and hGlu. The amino acids can be in either the L- or D-configuration. If nothing is specified it is understood that the amino acid residue is in the L configuration.

The expression “an amino acid residue having a neutral side chain” designates amino acid residues like Gly, Ala, Val, Leu, Ile, Phe, Pro, Ser, Thr, Cys, Met, Tyr, Asn and Gln.

When an insulin derivative according to the invention is stated to be “soluble at physiological pH values” it means that the insulin derivative can be used for preparing injectable insulin compositions that are fully dissolved at physiological pH values. Such favourable solubility may either be due to the inherent properties of the insulin derivative alone or a result of a favourable interaction between the insulin derivative and one or more ingredients contained in the vehicle.

The expression “high molar weight insulin” or “hmw” means that the molar weight of a complex of human insulin, of an insulin analogue or of an insulin derivative is above human serum albumin, above a dodecameric complex of an insulin analogue or of an insulin derivative or more than about 70 kDalton.

The expression “medium molar weight insulin” or “mmw” means that the molar weight of a complex of human insulin, of an insulin analogue or of an insulin derivative is from about an insulin hexamer to about an insulin dodecamer between 24 and 80 kDalton

The expression “low molar weight insulin” or “Imw” means that the molar weight of a human insulin, an insulin analogue or an insulin derivative is below 24 kDalton

The expression “net charge” means the overall charge of the molecule. At pH 7.4, human insulin has a negative net charge about −3 or when forming a hexamer about 2.5 per insulin monomer.

The following abbreviations have been used in the specification and examples:

hGlu homoglutamic acid

Sar: Sarcosine (N-methyl-glycine)

S.c. subcutaneous

Acyl ins Acylated insulin

Ins insulin

An “insulin” according to the invention is herein to be understood as human insulin, an insulin analogue and/or any combination thereof.

The term “human insulin” as used herein means the human hormone whose structure and properties are well-known. Human insulin has two polypeptide chains that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain, the second between the cysteine in position 7 of the A-chain and the cysteine in position 7 of the B-chain, and the third between the cysteine in position 20 of the A-chain and the cysteine in position 19 of the B-chain.

The hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains. By “insulin analogue” as used herein is meant a polypeptide derived from the primary structure of a naturally occurring insulin, for example that of human insulin, by mutation. One or more mutations are made by deleting and/or substituting at least one amino acid residue occurring in the naturally occurring insulin and/or by adding at least one amino acid residue. The added and/or substituted amino acid residues can either be codable amino acid residues or other naturally occurring amino acid residues.

In one embodiment an insulin analogue comprises less than 8 modifications (substitutions, deletions, additions and any combination thereof) relative to the parent insulin, alternatively less than 7 modifications relative to the parent insulin, alternatively less than 6 modifications relative to the parent insulin, alternatively less than 5 modifications relative to the parent insulin, alternatively less than 4 modifications relative to the parent insulin, alternatively less than 3 modifications relative to the parent insulin, alternatively less than 2 modifications relative to the parent insulin.

Mutations in the insulin molecule are denoted stating the chain (A or B), the position, and the three letter code for the amino acid substituting the native amino acid. By “desB30” or “B(1-29)” is meant a natural insulin B chain or analogue thereof lacking the B30 amino acid residue, and by B28Asp human insulin is meant human insulin wherein the amino acid residue in position 28 of the B chain has been substituted with Asp.

Degludec description page 4: The acylated insulin compounds of the present invention associate with each other to form complexes comprising zinc. These insulin-zinc complexes can be present in the pharmaceutical formulation as hexamers, dodecamers or complexes with a higher molar weight than dodecamers. All kinds of insulin form complexes with zinc, eg. human insulin, acylated insulin (insulin derivatives) and insulin analogues. In one embodiment of the invention at least 85% of the acylated insulin is present as complexes which are acylated insulin dodecamers or complexes with a higher molar weight than acylated insulin dodecamer.

In one embodiment of the invention at least 90, 92, 95, 96, 97, 98, 99 or 99.5% of the acylated insulin is present as complexes which are acylated insulin dodecamers or complexes with a higher molar weight than acylated insulin dodecamer.

In one embodiment of the invention, the pharmaceutical composition may comprise a surfactant. The surfactant may be present in an amount of 0.0005-0.01% based on the weight of the pharmaceutical composition. In one embodiment the surfactant can be present in an amount of 0.0005-0.007% based on the weight of the composition. An example of a surfactant could be polysorbate 20, which can be present in the composition in an amount of 0.001-0.003% based on the weight of the composition. Another example is poloxamer 188, which can be present in an amount of 0.002-0.006% based on the weight of the composition.

The long-acting insulin of the present invention may be acylated at various positions in the insulin molecule. In one embodiment, the long-acting insulin is acylated in the ε-amino group of a Lys residue in a position in the B-chain of the parent insulin molecule, for example, in the ε-amino group of the B29 lysine group in the human insulin molecule. However, according to other aspects of the invention the acylation may take place in another position in the long-acting insulin molecule, e.g. the α-amino group in position B1 or in position where the natural amino acid residue in the long-acting insulin molecule has been substituted with a lysine residue provided that B29 is changed from a lysine to another amino acid residue.

In one embodiment, the long-acting insulin is acylated either in the α-amino group in the B1 position or in a free ε-amino group of a lysine residue in the A- or B-chain of the insulin molecule.

In one embodiment, the long-acting insulin is acylated in the free ε-amino group of the lysine residue in position B29 of the insulin molecule.

The acyl group will be a lipophilic group and will typically be a fatty acid moiety having from about 6 to about 32 carbon atoms comprising at least one free carboxylic acid group or a group which is negatively charged at neutral pH. The fatty acid moiety will more typically have from 6 to 24, from 8 to 20, from 12 to 20, from 12-16, from 10-16, from 10-20, from 14-18 or from 14-16 carbon atoms.

In one embodiment, the pharmaceutical composition comprises at least one free carboxylic acid or a group which is negatively charged at neutral pH. In another embodiment, the pharmaceutical composition comprises an acyl group which is derived from a dicarboxylic fatty acid with from 4 to 32 carbon atoms.

In another embodiment, the fatty acid moiety is derived from a dicarboxylic fatty acid with from about 6 to about 32, from 6 to 24, from 8 to 20, from 12 to 20, from 12-16, from 10-16, from 10-20, from 14-18 or from 14-16 carbon atoms.

In one embodiment, the pharmaceutical composition comprises an acyl group which is attached to the insulin via a linker group through amide bonds.

The acyl group may be attached directly to the free amino group in question. However, the acyl group may also be attached via amide bonds by a linker which links the free amino group in the insulin molecule and the acyl group in question together.

The long-acting acylated insulin will typically have at least one, or two additional negative net charge compared to human insulin and more typically it will have two additional negative charges. The additional negative charge may be provided by the free carboxylic acid group in the fatty acid or by the linker group which may comprise one ore more amino acid residues of which at least one will contain a free carboxylic acid or a group which is negatively charged at neutral pH. In a further aspect the acyl group is derived from a dicarboxylic fatty acid.

In one embodiment, the pharmaceutical composition comprises long-acting insulin, wherein the insulin has a side chain attached either to the α-amino group of the N-terminal amino acid residue of the B chain or to an ε-amino group of a Lys residue present in the B chain of the parent insulin moiety via an amide bond, which side chain comprises at least one free carboxylic acid group or a group which is negatively charged at neutral pH, a fatty acid moiety with about 4 to about 32 carbon atoms in the carbon chain; and possibly one or more linkers linking the individual components in the side chain together via amide bonds.

In one embodiment, the long-acting insulin molecule has a side chain attached to the ε-amino group of a Lys residue present in the B chain of the parent insulin, the side chain being of the general formula:

—W—X—Y—Z₂

wherein W is:
an α-amino acid residue having a carboxylic acid group in the side chain which residue forms, with one of its carboxylic acid groups, an amide group together with ε-amino group of a Lys residue present in the B chain of the parent insulin;
a chain composed of two, three or four α-amino acid residues linked together via amide carbonyl bonds, which chain—via an amide bond—is linked to an ε-amino group of a Lys residue present in the B chain of the parent insulin, the amino acid residues of W being selected from the group of amino acid residues having a neutral side chain and amino acid residues having a carboxylic acid group in the side chain so that W has at least one amino acid residue which has a carboxylic acid group in the side chain; or
a covalent bond from X to an ε-amino group of a Lys residue present in the B chain of the parent insulin;

### X is:

—CO—;
—CH(COOH)CO—;
—CO—N(CH₂COOH)CH₂CO—;
—CO—N(CH₂COOH)CH₂CON(CH₂COOH)CH₂CO—;
—CO—N(CH₂CH₂COOH)CH₂CH₂CO—;
—CO—N(CH₂CH₂COOH)CH₂CH₂CON(CH₂CH₂COOH)CH₂CH₂CO—;
—CO—NHCH(COOH)(CH₂)₄NHCO—;
—CO—N(CH₂CH₂COOH)CH₂CO—; or
—CO—N(CH₂COOH)CH₂CH₂CO—.
   that
   a) when W is an amino acid residue or a chain of amino acid residues, via a bond from the underscored carbon forms an amide bond with an amino group in W, or
   b) when W is a covalent bond, via a bond from the underscored carbonyl carbon forms an amide bond with an ε-amino group of a Lys residue present in the B chain of the parent insulin;

### Y is:

—(CH₂)ₘ— where m is an integer in the range of 6 to 32;
a divalent hydrocarbon chain comprising 1, 2 or 3 —CH═CH— groups and a number of —CH₂— groups sufficient to give a total number of carbon atoms in the chain in the range of 10 to 32; and

### Z₂ is:

—COOH;
—CO-Asp;
—CO-Glu;
—CO-Gly;
—CO-Sar;
—CH(COOH)₂;
—N(CH₂COOH)₂;
—SO₃H; or
—PO₃H
   and any Zn²⁺ complexes thereof, provided that when W is a covalent bond and X is —CO—, then Z is different from —COOH.

In one embodiment of the present invention, the B30 amino acid residue has been deleted and the acylated insulin is a desB30 insulin.

In one embodiment of the present invention, W is an α-amino acid residue having from 4 to 10 carbon atoms and in a further aspect W is selected from the group consisting of α-Asp, β-Asp, α-Glu, γ-Glu, α-hGlu and δ-hGlu.

In one embodiment of the present invention, X is —CO—.

In one embodiment of the present invention, Z₂ is —COOH.

The substructure Y of the side chain —W—X—Y—Z₂ can be a group of the formula —(CH₂)ₘ— where m is an integer in the range of from 6 to 32, from 8 to 20, from 12 to 20, or from 12-16.

In one embodiment of the present invention, Y is a divalent hydrocarbon chain comprising 1, 2 or 3—CH═CH— groups and a number of —CH₂— groups sufficient to give a total number of carbon atoms in the chain in the range of from 6 to 32, from 10 to 32, from 12 to 20, or from 12-16.

In one embodiment of the present invention, Y is a divalent hydrocarbon chain of the formula —(CH₂)ᵥC₆H₄(CH₂)_{w}— wherein v and w are integers or one of them is zero so that the sum of v and w is in the range of from 6 to 30, from 10 to 20, or from 12-16.

In a further aspect W is selected from the group consisting of α-Asp, β-Asp, α-Glu, and γ-Glu; X is —CO— or —CH(COOH)CO; Y is —(CH₂)ₘ— where m is an integer in the range of 12-18 and Z₂ is —COOH or —CH(COOH)₂.

Non limiting examples of acylated insulin compounds are N^{εB29}—(N^{α}—(HOOC(CH₂)₁₄CO)-γ-Glu) desB30 human insulin; N^{εB29}—(N^{α}—(HOOC(CH₂)₁₅CO)-γ-Glu) desB30 human insulin; N^{εB29}—(N^{α}—(HOOC(CH₂)₁₆CO)-γ-Glu) desB30 human insulin; N^{εB29}—(N^{α}—(HOOC(CH₂)₁₇CO)-γ-Glu) desB30 human insulin; N^{εB29}—(N^{α}—(HOOC(CH₂)₁₈CO)-γ-Glu) desB30 human insulin; N^{εB29}—(N^{α}—(HOOC(CH₂)₁₆CO)-γ-Glu-N-(γ-Glu)) desB30 human insulin; N^{εB29}—(N^{α}-(Asp-OC(CH₂)₁₆CO)-γ-Glu) desB30 human insulin; N^{εB29}—(N^{α}-(Glu-OC(CH₂)₁₄CO)-γ-Glu) desB30 human insulin; N^{εB29}—(N^{α}-(Glu-OC(CH₂)₁₄CO—) desB30 human insulin; N^{εB29}—(N^{α}-(Asp-OC(CH₂)₁₆CO—) desB30 human insulin; N^{εB29}—(N^{α}—(HOOC(CH₂)₁₆CO)-α-Glu-N-(β-Asp)) desB30 human insulin; N^{εB29}—(N^{α}-(Gly-OC(CH₂)₁₃CO)-γ-Glu) desB30 human insulin; N^{εB29}—(N^{α}-(Sar-OC(CH₂)₁₃CO)-γ-Glu) desB30 human insulin; N^{εB29}—(N^{α}—(HOOC(CH₂)₁₃CO)-γ-Glu) desB30 human insulin; (N^{εB29}—(N^{α}—(HOOC(CH₂)₁₃CO)-β-Asp) desB30 human insulin; N^{εB29}—(N^{α}—(HOOC(CH₂)₁₃CO)-α-Glu) desB30 human insulin; N^{εB29}—(N^{α}—(HOOC(CH₂)₁₆CO)-γ-D-Glu) desB30 human insulin; N^{εB29}—(N^{α}—(HOOC(CH₂)₁₄CO)-β-D-Asp) desB30 human insulin N^{εB29}—(N^{α}—(HOOC(CH₂)₁₄CO)-β-D-Asp) desB30 human insulin; N^{εB29}—(N—HOOC(CH₂)₁₆CO-β-D-Asp) desB30 human insulin; N^{εB29}—(N—HOOC(CH₂)₁₄CO-IDA) desB30 human insulin; N^{εB29}—[N—(HOOC(CH₂)₁₆CO)—N-(carboxyethyl)-Gly] desB30 human insulin; N^{εB29}—[N—(HOOC(CH₂)₁₄CO)—N-(carboxyethyl)-Gly] desB30 human insulin; and N^{εB29}—[N—(HOOC(CH₂)₁₄CO)—N-(carboxymethyl)-β-Ala] desB30 human insulin.

In one embodiment of the present invention, the side chain may comprise at least one aromatic group or at least one dysfunctional PEG group. Hereinafter, the abbreviation “PEG” is used for polyethyleneglycol.

In one embodiment of the present invention, the acylated insulin used in the pharmaceutical composition is having a formula

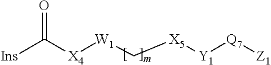

wherein Ins is the parent insulin moiety which via the α-amino group of the N-terminal amino acid residue of the B chain or an ε-amino group of a Lys residue present in the B chain of the insulin moiety is bound to the CO— group in the side chain via an amide bond;

### X₄ is

—(CH₂)ₙ where n is 1, 2, 3, 4, 5 or 6;
NR, where R is hydrogen or —(CH₂)ₚ—COOH; —(CH₂)ₚ—SO₃H; —(CH₂)ₚ—PO₃H₂, —(CH₂)ₚ—O—SO₃H₂; —(CH₂)ₚ—O—PO₃H₂; arylene substituted with 1 or 2 —(CH₂)ₚ—O—COOH groups; —(CH₂)ₚtetrazolyl, where p is an integer in the range of 1 to 6;
—(CR₁R₂)_{q}—NR—CO—, where R₁ and R₂ independently of each other and independently for each value of q can be H, —COOH, or OH, q is 1-6 and R is defined as above;
—((CR₃R₄)_{q1}—NR—CO)₂₋₄—, where R₃ and R₄ independently of each other and independently for each value of q₁ can be H, —COOH, or OH, q₁ is 1-6 and R is defined as above; or
a bond
   W₁ is arylene or heteroarylene, which may be substituted with one or two groups selected from the group consisting of —COOH, —SO₃H, and —PO₃H₂ and tetrazolyl, or W₁ is a bond;
   m is 0, 1, 2, 3, 4, 5 or 6;

### X₅ is

—O—;

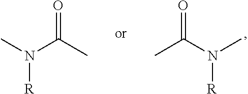

where R is defined as above; or
a bond;

### Y₁ is

—(CR₁R₂)_{q}—NR—CO—, where R₁ and R₂ independently of each other and independently for each value of q can be H, —COOH, a bond or OH, q is 1-6; and R is defined as above;
NR where R is defined as above;
—((CR₃R₄)_{q1}—NR—CO)₂₋₄—, where R₃ and R₄ independently of each other and independently for each value of q₁ can be H, —COOH, or OH, q₁ is 1-6 and R is defined as above; or
a bond;

### Q₇ is

—(CH₂)ᵣ— where r is an integer from 4 to 22;
a divalent hydrocarbon chain comprising 1, 2 or 3 —CH═CH— groups and a number of —CH₂— groups sufficient to give a total number of carbon atoms in the chain in the range of 4 to 22; or
a divalent hydrocarbon chain of the formula

—(CH₂)ₛ-Q₈-(C₆H₄)ᵥ₁-Q₉-(CH₂)_{w}-Q₁₀-(C₆H₄)ᵥ₂-Q₁₁-(CH₂)ₜ-Q₁₂-(C₆H₄)ᵥ₃-Q₁₃-(CH₂)_{z}—

wherein Q₈-Q₁₃ independently of each other can be O; S or a bond; where s, w, t and z independently of each other are zero or an integer from 1 to 10 so that the sum of s, w, t and z is in the range from 4 to 22, and v₁, v₂, and v₃ independently of each other can be zero or 1,
provided that when W₁ is a bond then Q₇ is not a divalent hydrocarbon chain of the formula —(CH₂)ᵥ₄C₆H₄(CH₂)_{W1}— wherein v₄ and w₁ are integers or one of them is zero so that the sum of v₄ and w₁ is in the range of 6 to 22; and

### Z₁ is:

—COOH;

—CO-Asp;

—CO-Glu;

—CO-Gly;

—CO-Sar;

—CH(COOH)₂;

—N(CH₂COOH)₂;

—SO₃H

—PO₃H₂;

—O—SO₃H;

—O—PO₃H₂;

-tetrazolyl or

—O—W₂,
where W₂ is arylene or heteroarylene substituted with one or two groups selected from —COOH, —SO₃H, and —PO₃H₂ and tetrazolyl;
provided that if W₁ is a bond and v₁, v₂ and v₃ are all zero and Q₁₋₆ are all a bond, then Z₁ is O—W₂
   and any Zn²⁺ complex thereof.

In one embodiment of the present invention, W₁ is phenylene. In another embodiment of the present invention, W₁ is 5-7 membered heterocyclic ring system comprising nitrogen, oxygen or sulphur. In another embodiment of the present invention, W₁ is a 5 membered heterocyclic ring system comprising at least one oxygen.

In one embodiment of the present invention, Q₇ is —(CH₂)ᵣ— where r is an integer in the range of from 4 to 22, from 8- to 20, from 12 to 20 or from 14-18. In one embodiment of the present invention, Q₈, Q₉, Q₁₂ and Q₁₃ are all bonds, v₂ is 1 and v₁ and v₃ are zero. In one embodiment of the present invention, Q₁₀ and Q₁₁ are oxygen.

In one embodiment of the present invention, X₄ and Y₁ are a bonds and X₅ is

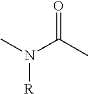

where R is —(CH₂)ₚ—COOH, where p is 1-4.

In one embodiment of the present invention, Z₁ is —COOH.

In one embodiment of the present invention, the acylated insulin of the pharmaceutical composition is selected from the group consisting of

0100-0000-0496 N^{εB29}—[N—(HOOC(CH₂)₁₄CO)—N-(carboxyethyl)-CH₂—C₆H₄CO] desB30 human insulin; 0100-0000-0515 N^{εB29}—[N—(HOOC(CH₂)₁₃CO)—N-(carboxyethyl)-CH₂—C₆H₄CO] desB30 human insulin; 0100-0000-0522 N^{εB29}—[N—(HOOC(CH₂)₁₅CO)—N-(carboxyethyl)-CH₂—C₆H₄CO] desB30 human insulin; 0100-0000-0488 N^{εB29}—[N—(HOOC(CH₂)₁₆CO)—N-(carboxyethyl)-CH₂—C₆H₄CO] desB30 human insulin; 0100-0000-0544 N^{εB29}—[N—(HOOC(CH₂)₁₄CO)—N-(carboxymethyl)-C₆H₄CO] desB30 human insulin, and 0100-0000-029 N^{εB29}—[N—(HOOC(CH₂)₁₄CO)—N-(carboxyethyl)-CH₂— (furanylene)CO] desB30 human insulin, 0100-0000-0552 N^{εB29}-{4-Carboxy-4-[10-(4-carboxy-phenoxy)-decanoylamino]-butyryl}desB30 human insulin

In one embodiment of the present invention, the acylated insulin present in the pharmaceutical composition is having a formula

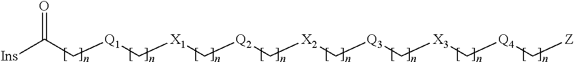

wherein Ins is the parent insulin moiety which via the α-amino group of the N-terminal amino acid residue of the B chain or an ε-amino group of a Lys residue present in the B chain of the insulin moiety is bound to the CO— group in the side chain via an amide bond;
each n is independently 0, 1, 2, 3, 4, 5 or 6;
Q₁, Q₂, Q₃, and Q₄ independently of each other can be
(CH₂CH₂O)ₛ—; (CH₂CH₂CH₂O)ₛ—; (CH₂CH₂CH₂CH₂O)ₛ—; (CH₂CH₂OCH₂CH₂CH₂CH₂O)ₛ— or (CH₂CH₂CH₂OCH₂CH₂CH₂CH₂O)ₛ— where s is 1-20
—(CH₂)ᵣ— where r is an integer from 4 to 22; or a divalent hydrocarbon chain comprising 1, 2 or 3 —CH═CH— groups and a number of —CH₂— groups sufficient to give a total number of carbon atoms in the chain in the range of 4 to 22;
—(CH₂)ₜ— or —(CH₂OCH₂)ₜ—, where t is an integer from 1 to 6;
—(CR₁R₂)_{q}—, where R₁ and R₂ independently of each other can be H, —COOH, (CH₂)₁₋₆COOH and R₁ and R₂ can be different at each carbon, and q is 1-6,
—((CR₃R₄)_{q1})₁—(NHCO—(CR₃R₄)_{q1}—NHCO)₁₋₂—((CR₃R₄)_{q1})₁ or —((CR₃R₄)_{q1})₁—(CONH—(CR₃R₄)_{q1}—CONH)₁₋₂—((CR₃R₄)_{q1}—)—, —((CR₃R₄)_{q1})₁—(NHCO—(CR₃R₄)_{q1}—CONH)₁₋₂—((CR₃R₄)_{q1})₁ or —((CR₃R₄)_{q1})₁—(CONH—(CR₃R₄)_{q1}—NHCO)₁₋₂—((CR₃R₄)_{q1})₁ where R₃ and R₄ independently of each other can be H, —COOH, and R₃ and R₄ can be different at each carbon, and q₁ is 1-6, or
a bond;

with the proviso that Q₁-Q₄ are different;

X₁, X₂ and X₃ are independently
O;
a bond; or

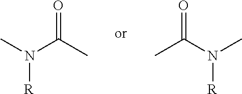

where R is hydrogen or —(CH₂)ₚ—COOH, —(CH₂)ₚ—SO₃H, —(CH₂)ₚ—PO₃H₂, —(CH₂)ₚ—O—SO₃H; —(CH₂)ₚ—O—PO₃H₂; or —(CH₂)ₚ-tetrazol-5-yl, where each p independently of the other p's is an integer in the range of 1 to 6; and

Z is:

—COOH;

—CO-Asp;

—CO-Glu;

—CO-Gly;

—CO-Sar;

—CH(COOH)₂,

—N(CH₂COOH)₂;

—SO₃H

—OSO₃H

—OPO3H₂

—PO₃H₂ or

-tetrazol-5-yl

and any Zn²⁺ complex thereof.

In one embodiment of the present invention, s is in the range of 2-12, 2-4 or 2-3. In one embodiment of the present invention, s is preferably 1.

In one embodiment of the present invention, Z is —COOH.

In one embodiment of the present invention, the acylated insulin of the pharmaceutical composition is selected from the group consisting of N^{εB29}-(3-[2-{2-(2-[ω-carboxypentadecanoyl-γ-glutamyl-(2-amino-ethoxy)]-ethoxy)-ethoxy}-ethoxy]-propinoyl) desB30 human insulin, N^{εB29}-(3-[2-{2-(2-[ω-carboxy-heptadecanoyl-γ-glutamyl-(2-amino-ethoxy)]-ethoxy)-ethoxy}-ethoxy]-propinoyl) desB30 human insulin, N^{εB29}-{3-[2-(2-{2-[2-(ω-carboxy-pentadecanoylamino)-ethoxy]-ethoxy}-ethoxy)-ethoxy]-propionyl-γ-glutamyl desB30 human insulin, N^{εB29}-(ω-[2-(2-{2-[2-(2-carboxy-ethoxy)-ethoxy]-ethoxy}-ethoxy)-ethylcarbamoyl]-heptadecanoyl-α-glutamyl) desB30 human insulin, N^{εB29}-(ω-[2-(2-{2-[2-(2-carboxy-ethoxy)ethoxy]-ethoxy}-ethoxy)-ethylcarbamoyl]-heptadecanoyl-γ-glutamyl) desB30 human insulin, N^{εB29}-3-[2-(2-{2-[2-(ω-carboxy-heptadecanoylamino)-ethoxy]-ethoxy}-ethoxy)-ethoxy]-propionyl-γ-glutamyl desB30 human insulin, N^{εB29}-(3-(3-{2-[2-(3-[7-carboxyheptanoylamino]propoxy)ethoxy]-ethoxy}propylcarbamoyl)propionyl) desB30 human insulin, N^{εB29}-(3-(3-{4-[3-(7-Carboxyheptanoylamino)propoxy]butoxy}propylcarbamoyl)propionyl-γ-glutamyl) desB30 human insulin, N^{εB29}-(3-(3-{2-[2-(3-[9-Carboxynonanoylamino]propoxy)ethoxy]ethoxy}-propylcarbamoyl)propionyl) desB30 human insulin, N^{εB29}-(3-(2-{2-[2-(9-carboxynonanoylamino)ethoxy]ethoxy}ethylcarbamoyl)propionyl-γ-glutamyl) desB30 human insulin, N^{εB29}-(3-(3-{4-[3-(9-Carboxynonanoylamino)propoxy]butoxy}-propylcarbamoyl)propionyl-γ-glutamyl) desB30 human insulin, N^{εB29}-(2-[3-(2-(2-{2-(7-carboxyheptanoylamino)ethoxy}ethoxy)ethylcarbamoyl]propionyl-γ-glutamyl) desB30 human insulin, N^{εB29}-(3-[2-(2-{2-[2-(ω-carboxypentadecanoylamino)ethoxy]ethoxy}ethoxy)ethoxy]propionyl)) desB30 human insulin, N^{εB29}-(3-(2-{2-[2-(2-{2-[2-(2-{2-[2-(2-{2-[2-(ω-carboxy-tridecanoylamino)-ethoxy]-ethoxy}-ethoxy)-ethoxy]-ethoxy}-ethoxy)-ethoxy]-ethoxy}-ethoxy)-ethoxy]-ethoxy}-ethoxy)-propionoyl-γ-glutamyl) desB30 human insulin, N^{εB29}-(3-[2-(2-{2-[2-(ω-Carboxy-tridecanoylamino)-ethoxy]-ethoxy}-ethoxy)-ethoxy]-propionoyl-γ-glutamyl) desB30 human insulin, N^{εB29}-(3-[2-(2-{2-[2-(2-{2-[2-(ω-carboxy-tridecanoylamino)-ethoxy]-ethoxy}-ethoxy)-ethoxy]-ethoxy}-ethoxy)-ethoxy]-ethoxy}-propionoyl-γ-glutamyl) desB30 human insulin, N^{εB29}-(3-(2-{2-[2-(ω-Carboxy-pentadecanoylamino)-ethoxy]-ethoxy}-ethylcarbamoyl)-propionyl-γ-glutamyl) desB30 human insulin, N^{εB29}-(3-(3-{2-[2-(3-[ω-Carboxypentadecanoylamino]propoxy)ethoxy]-ethoxy}propylcarbamoyl)propionyl-γ-glutamyl) desB30 human insulin, N^{εB29}-(3-(3-{4-[3-(ω-Carboxyundecanoylamino)propoxy]butoxypropylcarbamoyl)propionyl-γ-glutamyl) desB30 human insulin, N^{εB29}-(3-(3-{4-[3-(ω-carboxytridecanoylamino)propoxy]butoxypropylcarbamoyl)propionyl-γ-glutamyl) desB30 human insulin, N^{εB29}-(3-(2-{2-[2-(ω-Carboxyundecanoylamino)ethoxy]ethoxy}ethylcarbamoyl)propionyl-γ-glutamyl) desB30 human insulin, N^{εB29}-(3-(2-{2-[2-(ω-carboxytridecanoylamino)ethoxy]ethoxy}ethylcarbamoyl)propionyl-γ-glutamyl) desB30 human insulin, N^{εB29}-{3-[2-(2-{2-[2-(ω-carboxypentadecanoylamino)ethoxy]ethoxy]ethoxy)ethoxy}propionyl-gamma-γ-D-glutamyl) desB30 human insulin, N^{εB29}-{3-[2-(2-{2-[2-(7-carboxyheptanoylamino)ethoxy]ethoxy}ethoxy)ethoxy]-propionyl-γ-glutamyl} desB30 human insulin, N^{εB29}-{3-[2-(2-{2-[2-(9-carboxynonanoylamino)ethoxy]ethoxy}ethoxy)ethoxy]propionyl-γ-glutamyl} desB30 human insulin, N^{εB29}-{3-[2-(2-{2-[2-(ω-carboxyundecanoylamino)ethoxy]ethoxy}ethoxy)ethoxy]-propionyl-γ-glutamyl} desB30 human insulin, N^{εB29}-{3-[2-(2-{2-[2-(ω-carboxytridecanoylamino)ethoxy]ethoxy}ethoxy)ethoxy]propionyl-γ-glutamyl} desB30 human insulin.

The acylated insulins of the present invention may be produced as described in WO 2007/074133.

The parent insulin molecule is human insulin or an analogue thereof. Non-limiting analogues of human insulin is desB30 analogue; insulin analogues where the amino acid residue in position B30 is Lys and the amino acid residue in position B29 is any codable amino acid except Cys, Met, Arg and Lys; insulin analogues where the amino acid residue at position A21 is Asn and insulin analogues where the amino acid residue at position B3 is Lys and the amino acid residue at position B29 is Glu.

In another group of parent insulin analogues, the amino acid residue at position B28 is Asp. A specific example from this group of parent insulin analogues is AspB28 human insulin disclosed in EP 214826.

In another group of parent insulin analogues, the amino acid residue at position B28 is Lys and the amino acid residue at position B29 is Pro. A specific example from this group of parent insulin analogues is Lys^{B28}Pro^{B29} human insulin.

In another group of parent insulin analogues the amino acid residue in position B30 is Lys and the amino acid residue in position B29 is any codable amino acid except Cys, Met, Arg and Lys. An example is an insulin analogue where the amino acid residue at position B29 is Thr and the amino acid residue at position B30 is Lys. A specific example from this group of parent insulin analogues is Thr^{B29}Lys^{B30} human insulin.

In another group of parent insulin analogues, the amino acid residue at position B3 is Lys and the amino acid residue at position B29 is Glu. A specific example from this group of parent insulin analogues is Lys^{B3}Glu^{B29} human insulin. Examples of insulin analogues are such wherein Pro in position 28 of the B chain is mutated with Asp, Lys, Leu, Val, or Ala and/or Lys at position B29 is mutated with Pro, Glu or Asp. Furthermore, Asn at position B3 may be mutated with Thr, Lys, Gln, Glu or Asp. The amino acid residue in position A21 may be mutated with Gly. The amino acid in position B1 may be mutated with Glu. The amino acid in position B16 may be mutated with Glu or His. Further examples of insulin analogues are the deletion analogues e.g. analogues where the B30 amino acid in human insulin has been deleted (des(B30) human insulin), insulin analogues wherein the B1 amino acid in human insulin has been deleted (des(B1) human insulin), des(B28-B30) human insulin and des(B27) human insulin. Insulin analogues wherein the A-chain and/or the B-chain have an N-terminal extension and insulin analogues wherein the A-chain and/or the B-chain have a C-terminal extension such as with two arginine residues added to the C-terminal of the B-chain are also examples of insulin analogues. Further examples are insulin analogues comprising combinations of the mentioned mutations. Insulin analogues wherein the amino acid in position A14 is Asn, Gln, Glu, Arg, Asp, Gly or His, the amino acid in position B25 is His and which optionally further comprises one or more additional mutations are further examples of insulin analogues. Insulin analogues of human insulin wherein the amino acid residue in position A21 is Gly and wherein the insulin analogue is further extended in the C-terminal with two arginine residues are also examples of insulin analogues.

Further examples of insulin analogues include, but are not limited to: DesB30 human insulin; AspB28 human insulin; AspB28,desB30 human insulin; LysB3,GluB29 human insulin; LysB28,ProB29 human insulin; GluA14,HisB25 human insulin; HisA14,HisB25 human insulin; GluA14,HisB25,desB30 human insulin; HisA14, HisB25,desB30 human insulin; GluA14,HisB25,desB27,desB28,desB29,desB30 human insulin; GluA14,HisB25,GluB27,desB30 human insulin; GluA14,HisB16,HisB25,desB30 human insulin; HisA14,HisB16,HisB25,desB30 human insulin; HisA8,GluA14,HisB25,GluB27,desB30 human insulin; HisA8,GluA14,GluB1,GluB16,HisB25,GluB27,desB30 human insulin; and HisA8,GluA14,GluB16,HisB25,desB30 human insulin.

The pharmaceutical composition according to the present invention will comprise a therapeutically effective amount of the acylated insulin together with a pharmaceutically acceptable carrier for the treatment of type 1 diabetes, type 2 diabetes and other states that cause hyperglycaemia in patients in need of such a treatment.

In a further aspect of the invention, there is provided a pharmaceutical composition for treating type 1 diabetes, type 2 diabetes and other states that cause hyperglycaemia in a patient in need of such a treatment, comprising a therapeutically effective amount of an acylated insulin derivative as defined above in mixture with an insulin or an insulin analogue which has a rapid onset of action, together with pharmaceutically acceptable carriers and additives.

Thus the pharmaceutical composition may comprise a mixture of two insulin components: one with a protracted insulin action, a basal insulin, and the other with a rapid onset of action, a bolus insulin. An example of such mixture is Insulin aspart, AspB28 human insulin in mixture with N^{εB29}—(N^{α}—(HOOC(CH₂)₁₄CO)-γ-Glu) desB30 human insulin corresponding to LysB29Nε-hexadecandioyl-γ-Glu desB30 human insulin disclosed in WO 2005/012347. Another example of such a mixture is Lispro, Lys^{B28}Pro^{B29} human insulin, in mixture with LysB29Nε-hexadecandioyl-γ-Glu desB30 human insulin. A third example of such a mixture is Glulisine, Lys^{B3}Glu^{B29}-human insulin, in mixture with LysB29Nε-hexadecandioyl-γ-Glu desB30 human insulin.

In one embodiment of the present invention, at least 85% of the rapid acting insulin is present as rapid acting insulin hexamer or complexes with a smaller molar weight than rapid acting insulin hexamers.

In one embodiment of the present invention, at least 90, 92, 95, 96, 97, 98, 99, 99.5% of the rapid acting insulin is present as rapid acting insulin hexamer or complexes with a smaller molar weight than rapid acting insulin hexamers.

The acylated insulin derivative and the rapid acting insulin analogue can be mixed in a molar ratio about 90%/10%; about 75%/25%, about 70%/30% about 50%/50%, about 25%/75%, about 30%/70% or about 10%/90%.

In one embodiment the pharmaceutical composition according to the invention has a pH between about 6.5 to about 8.5. In another aspect the pH is from about 7.0 to about 8.2, the pH is from about 7.2 to 8.0 or from about 7.4 to about 8.0 or the pH is from about 7.4 to about 7.8.

The invention further comprises a method for producing a pharmaceutical composition comprising an acylated insulin wherein more than about 4 zinc atoms per 6 molecules of acylated insulin are added to the composition.

In a further aspect of the invention more than about 4.3 zinc atoms per 6 molecules of acylated insulin are added to the composition or more than about 4.5 zinc atoms per 6 molecules of acylated insulin are added to the composition or than about 5 zinc atoms per 6 molecules of acylated insulin are added to the composition. In a further aspect more than about 5.5 zinc atoms or more than about 6.5 zinc atoms, or more than about 7.0 zinc atoms or more than about 7.5 zinc atoms per 6 molecules of acylated insulin are added to the composition.

In one embodiment of the invention the method comprises adding up to about 12 zinc atoms per 6 molecules of acylated insulin to the composition.

In one embodiment of the invention the method comprises adding between about 4.3 and about 12 zinc atoms per 6 molecules of acylated insulin to the composition

In a further aspect of the invention between about 4.5 and about 12 zinc atoms per 6 molecules of acylated insulin are added to the composition or about 5 and about 11.4 zinc atoms per 6 molecules of acylated insulin are added to the composition or between about 5.5 and about 10 zinc atoms per 6 molecules of acylated insulin are added to the composition. In one embodiment of the present invention, the acylated insulin is selected from the group consisting of N^{εB29}—(N^{α}—(HOOC(CH₂)₁₄CO)-γ-Glu) desB30 human insulin; N^{εB29}—(N^{α}—(HOOC(CH₂)₁₅CO)-γ-Glu) desB30 human insulin; N^{εB29}—(N^{α}—(HOOC(CH₂)₁₆CO)-γ-Glu) desB30 human insulin; N^{εB29}—(N^{α}—(HOOC(CH₂)₁₇CO)-γ-Glu) desB30 human insulin; N^{εB29}—(N^{α}—(HOOC(CH₂)₁₈CO)-γ-Glu) desB30 human insulin; N^{εB29}—(N^{α}—(HOOC(CH₂)₁₆CO)-γ-Glu-N-(γ-Glu)) desB30 human insulin; N^{εB29}—(N^{α}-(Asp-OC(CH₂)₁₆CO)-γ-Glu) desB30 human insulin; N^{εB29}—(N^{α}-(Glu-OC(CH₂)₁₄CO)-γ-Glu) desB30 human insulin; N^{εB29}—(N^{α}-(Glu-OC(CH₂)₁₄CO—) desB30 human insulin; N^{εB29}—(N^{α}-(Asp-OC(CH₂)₁₆CO—) desB30 human insulin; N^{εB29}—(N^{α}-(HOOC(CH₂)₁₆CO)-α-Glu-N-(β-Asp)) desB30 human insulin; N^{εB29}—(N^{α}-(Gly-OC(CH₂)₁₃CO)-γ-Glu) desB30 human insulin; N^{εB29}—(N^{α}-(Sar-OC(CH₂)₁₃CO)-γ-Glu) desB30 human insulin; N^{εB29}—(N^{α}—(HOOC(CH₂)₁₃CO)-γ-Glu) desB30 human insulin; (N^{εB29}—(N^{α}—(HOOC(CH₂)₁₃CO)-β-Asp) desB30 human insulin; N^{εB29}—(N^{α}—(HOOC(CH₂)₁₃CO)-α-Glu) desB30 human insulin; N^{εB29}—(N^{α}—(HOOC(CH₂)₁₆CO)-γ-D-Glu) desB30 human insulin; N^{εB29}—(N^{α}—(HOOC(CH₂)₁₄CO)-β-D-Asp) desB30 human insulin N^{εB29}—(N^{α}—(HOOC(CH₂)₁₄CO)-β-D-Asp) desB30 human insulin; N^{εB29}—(N—HOOC(CH₂)₁₆CO-β-D-Asp) desB30 human insulin; N^{εB29}—(N—HOOC(CH₂)₁₄CO-IDA) desB30 human insulin; N^{εB29}—[N—(HOOC(CH₂)₁₆CO)—N-(carboxyethyl)-Gly] desB30 human insulin; N^{εB29}—[N—(HOOC(CH₂)₁₄CO)—N-(carboxyethyl)-Gly] desB30 human insulin; and N^{εB29}—[N—(HOOC(CH₂)₁₄CO)—N-(carboxymethyl)-β-Ala] desB30 human insulin.

The term “nicotinic compound” includes nicotinamide, nicotinic acid, niacin, niacin amide and vitamin B3 and/or salts thereof and/or any combination thereof.

According to the present invention, the concentration of the nicotinic compound and/or salts thereof is in the range from about 1 mM to about 300 mM or from about 5 mM to about 200 mM.

The term “arginine” or “Arg” includes the amino acid arginine and/or a salt thereof.

In one embodiment, the insulin preparation comprises 1 to 100 mM of arginine.

In one embodiment, the insulin preparation comprises 1 to 20 mM of arginine.

In one embodiment, the insulin preparation comprises 20 to 90 mM of arginine.

In one embodiment, the insulin preparation comprises 30 to 85 mM of arginine.

The term “pharmaceutical preparation” or “insulin preparation” as used herein means a product comprising a fast acting insulin compound, a long-acting insulin compound, a nicotinic compound and an aminoacid, optionally together with other excipients such as preservatives, chelating agents, tonicity modifiers, bulking agents, stabilizers, antioxidants, polymers and surfactants, metal ions, oleaginous vehicles and proteins (e.g., human serum albumin, gelatine or proteins), said insulin preparation being useful for treating, preventing or reducing the severity of a disease or disorder by administration of said insulin preparation to a person. Thus, an insulin preparation is also known in the art as a pharmaceutical preparation, a pharmaceutical composition or composition.

The buffer may be selected from the group consisting of, but not limited to, sodium acetate, sodium carbonate, citrate, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

The insulin preparation of the present invention may further comprise other ingredients common to insulin preparations, for example zinc complexing agents such as citrate, and phosphate buffers.

Glycerol and/or mannitol and/or sodium chloride may be present in an amount corresponding to a concentration of 0 to 250 mM, 0 to 200 mM or 0 to 100 mM.

Stabilizers, surfactants and preservatives may also be present in the insulin preparations of this invention.

The insulin preparations of the present invention may further comprise a pharmaceutically acceptable preservative. The preservative may be present in an amount sufficient to obtain a preserving effect. The amount of preservative in an insulin preparation may be determined from e.g. literature in the field and/or the known amount(s) of preservative in e.g. commercial products. Each one of these specific preservatives constitutes an alternative embodiment of the invention. The use of a preservative in pharmaceutical preparations is described, for example in Remington: The Science and Practice of Pharmacy, 19^{th} edition, 1995.

The preservative present in the insulin preparation of this invention may be as in the heretofore conventional insulin preparations, for example phenol, m-cresol and methylparaben.

The insulin preparation of the present invention may further comprise a chelating agent. The use of a chelating agent in pharmaceutical preparations is well-known to the skilled person. For convenience reference is made to Remington: The Science and Practice of Pharmacy, 19^{th} edition, 1995.

The insulin preparation of the present invention may further comprise a stabilizer. The term “stabilizer” as used herein refers to chemicals added to polypeptide containing pharmaceutical preparations in order to stabilize the peptide, i.e. to increase the shelf life and/or in-use time of such preparations. For convenience reference is made to Remington: The Science and Practice of Pharmacy, 19^{th} edition, 1995.

The insulin preparation of the present invention may further comprise a surfactant. The term “surfactant” as used herein refers to any molecules or ions that are comprised of a water-soluble (hydrophilic) part, the head, and a fat-soluble (lipophilic) segment. Surfactants accumulate preferably at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The term “detergent” is a synonym used for surfactants in general. The use of a surfactant in pharmaceutical preparations is well-known to the skilled person. For convenience reference is made to Remington: The Science and Practice of Pharmacy, 19^{th} edition, 1995.

In a further embodiment the invention relates to an insulin preparation comprising an aqueous solution of an insulin compound of the present invention, and a buffer, wherein said insulin compound is present in a concentration from 0.1 mM or above, and wherein said preparation has a pH from about 3.0 to about 8.5 at room temperature (˜25° C.).

The present invention also relates to methods for producing the insulin preparations of the invention.

In one embodiment, the method for making insulin preparations of the invention comprises:

a) preparing a solution by dissolving the insulin compounds separately or a mixture of insulin compounds in water or buffer;

b) preparing a solution by dissolving a divalent metal ion in water or buffer;

c) preparing a solution by dissolving one, two or more preservatives in water or buffer; or dissolving the preservatives separately in water or buffer

d) preparing a solution by dissolving an isotonicity agent in water or buffer;

e) preparing a solution by dissolving a buffer in water

f) preparing a solution by dissolving a surfactant and/or a stabilizer in water or buffer;

g) preparing a solution by dissolving nicotinamide in water or buffer

h) mixing solution a) and one or more of solutions b), c), d), e), f) and g));

Finally adjusting the pH of the mixture in h) to the desired pH followed by a sterile filtration.

In one embodiment, the method for making insulin preparations of the invention comprises:

a) preparing a solution by dissolving the insulin compounds separately or a mixture of insulin compounds in water or buffer;

b) preparing a solution by dissolving a divalent metal ion in water or buffer;

c) preparing a solution by dissolving one, two or more preservatives in water or buffer; or dissolving the preservatives separately in water or buffer

d) preparing a solution by dissolving an isotonicity agent in water or buffer;

e) preparing a solution by dissolving a buffer in water

f) preparing a solution by dissolving a surfactant and/or a stabilizer in water or buffer;

g) preparing a solution by dissolving an absorption rate enhancer in water or buffer

h) mixing solution a) and one or more of solutions b), c), d), e), f) and g));

Finally adjusting the pH of the mixture in h) to the desired pH followed by a sterile filtration.

In one embodiment, the method for making insulin preparations of the invention comprises the following sequential steps:

a) preparing a solution with the long-acting insulin compound adding one, two or more phenolic preservatives and eventually isotonicity agent, buffer, stabilizer, and nicotinamide to a) before addition of zinc,

c) adding zinc about pH 7.4 or above,

d) waiting 1 hour to overnight if addition of zinc occurred at pH 7.4 or down to few minutes if pH at zinc addition was about 7.8,

e) adding the rapid-acting insulin compound and

f) adding nicotinamide.

In one embodiment, the method for making insulin preparations of the invention comprises the following sequential steps:

a) preparing a solution with insulin degludec

b) adding a phenol and m-cresol to a) before addition of zinc,

c) adding zinc at pH 7.4 or above,

d) verifying that all degludec is in di-hexamer form

d) waiting 1 hour to overnight if addition of zinc occurred at pH 7.4 or down to few minutes if pH about 7.8,

e) adding insulin aspart and

f) adding nicotinamide.

In one embodiment, the method for making insulin preparations of the invention comprises the following sequential steps:

a) preparing a solution with insulin detemir

b) preparing a solution by dissolving zinc acetate or chloride in water or buffer;

c) preparing a solution by dissolving a preservative in water or buffer;

d) preparing a solution by dissolving an isotonicity agent in water or buffer;

e) preparing a solution by dissolving a surfactant and/or a stabilizer in water or buffer;

f) mixing solution a) and one or more of solutions b), c), d), and e);

g) preparing a solution of insulin aspart;

h) mixing g) with b) to about 3 Zn/6ins, adding preservative solutions and adjust pH to 7.4;

h) mixing detemir solution including zinc and preservatives with aspart solution including zinc and preservatives;

i) addition of nicotinamide.

Finally adjusting the pH of the mixture in i) to the desired pH followed by a sterile filtration.

The term “absorption rate enhancer” as used herein means a substance which increases the absorption rate from a subcutaneous depot into the blood. Examples of absorption rate enhancers are nicotinamide, hyaluronidase, EDTA (edetate) and citrate.

The insulin preparations of the present invention can be used in the treatment of diabetes by parenteral administration. It is recommended that the dosage of the insulin preparations of this invention which is to be administered to the patient be selected by a physician.

Parenteral administration may be performed by subcutaneous, intramuscular, intraperitoneal or intravenous injection by means of a syringe, optionally a pen-like syringe. Alternatively, parenteral administration can be performed by means of an infusion pump. As a further option, the insulin preparations containing the insulin compound of the invention can also be adapted to transdermal administration, e.g. by needle-free injection or from a patch, optionally an iontophoretic patch, or transmucosal, e.g. buccal, administration.

Insulin preparations according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

The insulin preparations of the present invention may be administered simultaneously or sequentially with OAD(s) or GLP-1. The factors may be supplied in single-dosage form wherein the single-dosage form contains both compounds, or in the form of a kit-of-parts comprising a preparation of a the pharmaceutical composition comprising a pharmaceutical composition comprising an acylated insulin and a pharmaceutical composition containing an OAD as a second unit dosage form. Whenever a first or second or third, etc., unit dose is mentioned throughout this specification this does not indicate the preferred order of administration, but is merely done for convenience purposes.

By “simultaneous” dosing of a preparation of a pharmaceutical composition comprising an acylated insulin and a preparation of OAD(s) or GLP-1 is meant administration of the compounds in single-dosage form, or administration of a first agent followed by administration of a second agent with a time separation of no more than 15 minutes, 10, 5 or 2 minutes. Either factor may be administered first.

By “sequential” dosing is meant administration of a first agent followed by administration of a second agent with a time separation of more than 15 minutes. Either of the two unit dosage form may be administered first. Preferably, both products are injected through the same intravenous access.

In one embodiment of the present invention, the insulin preparation is administered once daily simultaneously or sequentially with OAD(s) or GLP-1. In another embodiment of the present invention, the insulin preparation can be given up to 5 times daily.

In one embodiment of the invention the insulin preparation is an aqueous preparation, i.e. preparation comprising water. Such preparation is typically a solution or a suspension. In a further embodiment of the invention the insulin preparation is an aqueous solution.

The term “aqueous preparation” is defined as a preparation comprising at least 50% w/w water. Likewise, the term “aqueous solution” is defined as a solution comprising at least 50% w/w water, and the term “aqueous suspension” is defined as a suspension comprising at least 50% w/w water.

Aqueous suspensions may contain the active compounds in admixture with excipients suitable for the manufacture of aqueous suspensions.

In one embodiment, the insulin preparations of this invention are well-suited for application in pen-like devices used for insulin therapy by injection.

The term “physical stability” of the insulin preparation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein preparations is evaluated by means of visual inspection and/or turbidity measurements after exposing the preparation filled in suitable containers (e.g. cartridges or vials) to mechanical/physical stress (e.g. agitation) at different temperatures for various time periods. Visual inspection of the preparations is performed in a sharp focused light with a dark background. The turbidity of the preparation is characterized by a visual score ranking the degree of turbidity for instance on a scale from 0 to 3 (a preparation showing no turbidity corresponds to a visual score 0, and a preparation showing visual turbidity in daylight corresponds to visual score 3). A preparation is classified physically unstable with respect to protein aggregation, when it shows visual turbidity in daylight. Alternatively, the turbidity of the preparation can be evaluated by simple turbidity measurements well-known to the skilled person. Physical stability of the aqueous protein preparations can also be evaluated by using a spectroscopic agent or probe of the conformational status of the protein. The probe is preferably a small molecule that preferentially binds to a non-native conformer of the protein. One example of a small molar spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

The term “chemical stability” of the protein preparation as used herein refers to changes in the covalent protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Increasing amounts of chemical degradation products is often seen during storage and use of the protein preparation. Most proteins are prone to deamidation, a process in which the side chain amide group in glutaminyl or asparaginyl residues is hydrolysed to form a free carboxylic acid or asparaginyl residues to form an IsoAsp derivative. Other degradations pathways involves formation of high molecular weight products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein preparation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC). Since HMWP products are potentially immunogenic and not biologically active, low levels of HMWP are advantageous.

The term “stabilized preparation” refers to a preparation with increased physical stability, increased chemical stability or increased physical and chemical stability. In general, a preparation must be stable during use and storage (in compliance with recommended use and storage conditions) until the expiration date is reached.

The term “diabetes” or “diabetes mellitus” includes type 1 diabetes, type 2 diabetes, gestational diabetes (during pregnancy) and other states that cause hyperglycaemia. The term is used for a metabolic disorder in which the pancreas produces insufficient amounts of insulin, or in which the cells of the body fail to respond appropriately to insulin thus preventing cells from absorbing glucose. As a result, glucose builds up in the blood.

Type 1 diabetes, also called insulin-dependent diabetes mellitus (IDDM) and juvenile-onset diabetes, is caused by B-cell destruction, usually leading to absolute insulin deficiency.

Type 2 diabetes, also known as non-insulin-dependent diabetes mellitus (NIDDM) and adult-onset diabetes, is associated with predominant insulin resistance and thus relative insulin deficiency and/or a predominantly insulin secretory defect with insulin resistance.

The term “pharmaceutically acceptable” as used herein means suited for normal pharmaceutical applications, i.e., not giving rise to any serious adverse events in patients.

The term “treatment of a disease” as used herein means the management and care of a patient having developed the disease, condition or disorder and includes treatment, prevention or alleviation of the disease. The purpose of treatment is to combat the disease, condition or disorder. Treatment includes the administration of the active compounds to eliminate or control the disease, condition or disorder as well as to alleviate the symptoms or complications associated with the disease, condition or disorder, and prevention of the disease, condition or disorder.

In its broadest sense, the term a “critically ill patient”, as used herein refers to a patient who has sustained or are at risk of sustaining acutely life-threatening single or multiple organ system failure due to disease or injury, a patient who is being operated and where complications supervene, and a patient who has been operated in a vital organ within the last week or has been subject to major surgery within the last week. In a more restricted sense, the term a “critically ill patient”, as used herein refers to a patient who has sustained or are at risk of sustaining acutely life-threatening single or multiple organ system failure due to disease or injury, or a patient who is being operated and where complications supervene. In an even more restricted sense, the term a “critically ill patient”, as used herein refers to a patient who has sustained or are at risk of sustaining acutely life-threatening single or multiple organ system failure due to disease or injury. Similarly, these definitions apply to similar expressions such as “critical illness in a patient” and a “patient is critically ill”. Examples of a critically ill patient is a patient in need of cardiac surgery, cerebral surgery, thoracic surgery, abdominal surgery, vascular surgery, or transplantation, or a patient suffering from neurological diseases, cerebral trauma, respiratory insufficiency, abdominal peritonitis, multiple trauma or severe burns, or critical illness polyneuropathy.

The term “anabolism” as used herein, means the set of metabolic pathways that construct molecules from smaller units. These reactions require energy. One way of categorizing metabolic processes, whether at the cellular, organ or organism level is as ‘anabolic’ or as ‘catabolic’, which is the opposite. Anabolism is powered by catabolism, where large molecules are broken down into smaller parts and then used up in respiration. Many anabolic processes are powered by adenosine triphosphate (ATP). Anabolic processes tend toward “building up” organs and tissues. These processes produce growth and differentiation of cells and increase in body size, a process that involves synthesis of complex molecules. Examples of anabolic processes include the growth and mineralization of bone and increases in muscle mass. Endocrinologists have traditionally classified hormones as anabolic or catabolic, depending on which part of metabolism they stimulate. The balance between anabolism and catabolism is also regulated by circadian rhythms, with processes such as glucose metabolism fluctuating to match an animal's normal periods of activity throughout the day. Some examples of the “anabolic effects” of these hormones are increased protein synthesis from amino acids, increased appetite, increased bone remodeling and growth, and stimulation of bone marrow, which increases the production of red blood cells. Through a number of mechanisms anabolic hormones stimulate the formation of muscle cells and hence cause an increase in the size of skeletal muscles, leading to increased strength.

In another embodiment, an insulin analogue according to the invention is used as a medicament for delaying or preventing disease progression in type 2 diabetes.

In one embodiment of the present invention, the insulin preparation according to the invention is for use as a medicament for the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, stroke, coronary heart disease and other cardiovascular disorders is provided.

In a further embodiment of the present invention, a method for the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, coronary heart disease and other cardiovascular disorders, stroke, the method comprising administering to a patient in need of such treatment an effective amount for such treatment of an insulin preparation according to the invention, is provided.

The treatment with an insulin preparation according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity.

The treatment with an insulin preparation according to the present invention may also be combined with bariatric surgery—a surgery that influences the glucose levels and/or lipid homeostasis such as gastric banding or gastric bypass.

The production of polypeptides, e.g., insulins, is well known in the art. An insulin compound according to the invention may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The insulin compound may also be produced by a method which comprises culturing a host cell containing a DNA sequence encoding the analogue and capable of expressing the insulin compound in a suitable nutrient medium under conditions permitting the expression of the insulin compound. For insulin compound comprising non-natural amino acid residues, the recombinant cell should be modified such that the non-natural amino acids are incorporated into the compound, for instance by use of tRNA mutants. Hence, briefly, the insulin compounds according to the invention are prepared analogously to the preparation of known insulin compounds.

Several methods may be used for the production of insulin compounds. For example three major methods which are used in the production of insulin in microorganisms are disclosed in WO2008034881. Two of these involve Escherichia coli, with either the expression of a large fusion protein in the cytoplasm (Frank et al. (1981) in Peptides: Proceedings of the 7^{th} American Peptide Chemistry Symposium (Rich & Gross, eds.), Pierce Chemical Co., Rockford, Ill. pp 729-739), or use of a signal peptide to enable secretion into the periplasmic space (Chan et al. (1981) PNAS 78:5401-5404). A third method utilizes Saccharomyces cerevisiae to secrete an insulin precursor into the medium (Thim et al. (1986) PNAS 83:6766-6770). The prior art discloses a number of insulin precursors which are expressed in either E. coli or Saccharomyces cerevisiae, vide U.S. Pat. No. 5,962,267, WO 95/16708, EP 0055945, EP 0163529, EP 0347845 and EP 0741188.

The insulin compounds are produced by expressing a DNA sequence encoding the insulin compound in question in a suitable host cell by well known technique as disclosed in e.g. U.S. Pat. No. 6,500,645. The insulin compound is either expressed directly or as a precursor molecule which has an N-terminal extension on the B-chain or a C-terminal extension on the B-chain. The N-terminal extension may have the function of increasing the yield of the directly expressed product and may be of up to 15 amino acid residues long. The N-terminal extension is to be cleaved of in vitro after isolation from the culture broth and will therefore have a cleavage site next to B1. N-terminal extensions of the type suitable in the present invention are disclosed in U.S. Pat. No. 5,395,922, and EP 765,395. The C-terminal extension may have the function of protecting the mature insulin or insulin analogue molecule against intracellular proteolytic processing by host cell exoproteases. The C-terminal extension is to be cleaved of either extra-cellularly in the culture broth by secreted, active carboxypeptidase or in vitro after isolation from the culture broth. A method for producing mature insulin and insulin compound with C-terminal extensions on the B-chain that are removed by carboxypetidase are disclosed in WO 08037735. The target insulin product of the process may either be a two-chain human insulin or a two-chain human insulin analogue which may or may not have a short C-terminal extension of the B-chain. If the target insulin product will have no C-terminal extension of the B-chain, then said C-terminal extension should be capable of subsequently being cleaved off from the B-chain before further purification steps.

The present invention also contemplates the following non-limiting list of embodiments, which are further described elsewhere herein:
1. An insulin preparation comprising:
   an acylated insulin or an analog thereof,
   human insulin or an insulin analog,
   a nicotinic compound, and
   arginine.
2. The insulin preparation according to embodiment 1, wherein the acylated insulin or an analog thereof is an insulin acylated in the ε-amino group of a Lys residue in a position in the B-chain of the parent insulin molecule.
3. The insulin preparation according to any one of the previous embodiments, wherein the acyl group of the acylated insulin or an analog thereof comprises at least one free carboxylic acid or a group which is negatively charged at neutral pH.
4. The insulin preparation according to any one of the previous embodiments, wherein the acyl group of the acylated insulin or an analog thereof is derived from a dicarboxylic fatty acid with from 4 to 32 carbon atoms.
5. The insulin preparation according to any one of embodiments 1 or 5, wherein the acyl group of the acylated insulin or an analog thereof is attached to the insulin molecule via a linker group through amide bonds.
6. The insulin preparation according to any one of embodiments 1 or 6, wherein the linker group comprises at least one free carboxylic group or a group which is negatively charged at neutral pH.
7. The insulin preparation according to any one of the previous embodiments, wherein the insulin molecule has a side chain attached either to the α-amino group of the N-terminal amino acid residue of the B chain or to an ε-amino group of a Lys residue present in the B chain of the parent insulin moiety via an amide bond, which side chain comprises at least one free carboxylic acid group or a group which is negatively charged at neutral pH, a fatty acid moiety with about 4 to about 32 carbon atoms in the carbon chain; and possible one or more linkers linking the individual components in the side chain together via amide bonds.
8. The insulin preparation according to any one of the previous embodiments, wherein the side chain comprises at least one aromatic group.
9. The insulin preparation according to any one of embodiments 1-7, wherein the side chain comprises at least one difunctional PEG group.
10. The insulin preparation according to any one of embodiments 1-7, wherein the insulin molecule has a side chain attached to the ε-amino group of a Lys residue present in the B chain of the parent insulin, the side chain being of the general formula:

—W—X—Y—Z₂

wherein W is:
an α-amino acid residue having a carboxylic acid group in the side chain which residue forms, with one of its carboxylic acid groups, an amide group together with ε-amino group of a Lys residue present in the B chain of the parent insulin;
a chain composed of two, three or four α-amino acid residues linked together via amide carbonyl bonds, which chain—via an amide bond—is linked to an ε-amino group of a Lys residue present in the B chain of the parent insulin, the amino acid residues of W being selected from the group of amino acid residues having a neutral side chain and amino acid residues having a carboxylic acid group in the side chain so that W has at least one amino acid residue which has a carboxylic acid group in the side chain; or
a covalent bond from X to an ε-amino group of a Lys residue present in the B chain of the parent insulin;

### X is:

—CO—;
—CH(COOH)CO—;
—CO—N(CH₂COOH)CH₂CO—;
—CO—N(CH₂COOH)CH₂CON(CH₂COOH)CH₂CO—;
—CO—N(CH₂CH₂COOH)CH₂CH₂CO—;
—CO—N(CH₂CH₂COOH)CH₂CH₂CON(CH₂CH₂COOH)CH₂CH₂CO—;
—CO—NHCH(COOH)(CH₂)₄NHCO—;
—CO—N(CH₂CH₂COOH)CH₂CO—; or
—CO—N(CH₂COOH)CH₂CH₂CO—.
   that
   a) when W is an amino acid residue or a chain of amino acid residues, via a bond from the underscored carbon forms an amide bond with an amino group in W, or
   b) when W is a covalent bond, via a bond from the underscored carbonyl carbon forms an amide bond with an ε-amino group of a Lys residue present in the B chain of the parent insulin;

### Y is:

—(CH₂)ₘ— where m is an integer in the range of 6 to 32;
a divalent hydrocarbon chain comprising 1, 2 or 3 —CH═CH— groups and a number of —CH₂— groups sufficient to give a total number of carbon atoms in the chain in the range of 10 to 32; and

### Z₂ is:

—COOH;
—CO-Asp;
—CO-Glu;
—CO-Gly;
—CO-Sar;
—CH(COOH)₂;
—N(CH₂COOH)₂;
—SO₃H; or
—PO₃H
   and any Zn²⁺ complexes thereof, provided that when W is a covalent bond and X is —CO—, then Z is different from —COOH.
11. The insulin preparation according to any one of embodiments 1-7 and 10, wherein Z₂ is —COOH.
12. The insulin preparation according to any one of embodiments 1-7 and 10-11, wherein the acylated insulin is selected from the group consisting of N^{εB29}—(N^{α}—(HOOC(CH₂)₁₄CO)-γ-Glu) desB30 human insulin; N^{εB29}—(N^{α}—(HOOC(CH₂)₁₅CO)-γ-Glu) desB30 human insulin; N^{εB29}—(N^{α}—(HOOC(CH₂)₁₆CO)-γ-Glu) desB30 human insulin; N^{εB29}—(N^{α}—(HOOC(CH₂)₁₇CO)-γ-Glu) desB30 human insulin; N^{εB29}—(N^{α}—(HOOC(CH₂)₁₈CO)-γ-Glu) desB30 human insulin; N^{εB29}—(N^{α}—(HOOC(CH₂)₁₆CO)-γ-Glu-N-(γ-Glu)) desB30 human insulin; N^{εB29}—(N^{α}-(Asp-OC(CH₂)₁₆CO)-γ-Glu) desB30 human insulin; N^{εB29}—(N^{α}-(Glu-OC(CH₂)₁₄CO)-γ-Glu) desB30 human insulin; N^{εB29}—(N^{α}-(Glu-OC(CH₂)₁₄CO—) desB30 human insulin; N^{εB29}—(N^{α}-(Asp-OC(CH₂)₁₆CO—) desB30 human insulin; N^{εB29}—(N^{α}—(HOOC(CH₂)₁₆CO)-α-Glu-N-(β-Asp)) desB30 human insulin; N^{εB29}—(N^{a}-(Gly-OC(CH₂)₁₃CO)-γ-Glu) desB30 human insulin; N^{εB29}—(N^{α}-(Sar-OC(CH₂)₁₃CO)-γ-Glu) desB30 human insulin; N^{εB29}—(N^{α}—(HOOC(CH₂)₁₃CO)-γ-Glu) desB30 human insulin; (N^{εB29}—(N^{α}—(HOOC(CH₂)₁₃CO)-β-Asp) desB30 human insulin; N^{εB29}—(N^{α}—(HOOC(CH₂)₁₃CO)-α-Glu) desB30 human insulin; N^{εB29}—(N^{α}—(HOOC(CH₂)₁₆CO)-γ-D-Glu) desB30 human insulin; N^{εB29}—(N^{α}—(HOOC(CH₂)₁₄CO)-β-D-Asp) desB30 human insulin N^{εB29}—(N^{α}—(HOOC(CH₂)₁₄CO)-β-D-Asp) desB30 human insulin; N^{εB29}—(N—HOOC(CH₂)₁₆CO-β-D-Asp) desB30 human insulin; N^{εB29}—(N—HOOC(CH₂)₁₄CO-IDA) desB30 human insulin; N^{εB29}—[N—(HOOC(CH₂)₁₆CO)—N-(carboxyethyl)-Gly] desB30 human insulin; N^{εB29}—[N—(HOOC(CH₂)₁₄CO)—N-(carboxyethyl)-Gly] desB30 human insulin; and N^{εB29}—[N—(HOOC(CH₂)₁₄CO)—N-(carboxymethyl)-β-Ala] desB30 human insulin.
13. The insulin preparation according to any one of embodiments 1-8, wherein the acylated insulin is having a formula

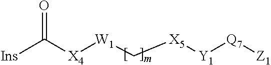

wherein Ins is the parent insulin moiety which via the α-amino group of the N-terminal amino acid residue of the B chain or an ε-amino group of a Lys residue present in the B chain of the insulin moiety is bound to the CO— group in the side chain via an amide bond;

### X₄ is

—(CH₂)ₙ where n is 1, 2, 3, 4, 5 or 6;
NR, where R is hydrogen or —(CH₂)ₚ—COOH; —(CH₂)ₚ—SO₃H; —(CH₂)ₚ—PO₃H₂, —(CH₂)ₚ—O—SO₃H₂; —(CH₂)ₚ—O—PO₃H₂; arylene substituted with 1 or 2 —(CH₂)ₚ—O—COOH groups; —(CH₂)ₚ-tetrazolyl, where p is an integer in the range of 1 to 6;
—(CR₁R₂)_{q}—NR—CO—, where R₁ and R₂ independently of each other and independently for each value of q can be H, —COOH, or OH, q is 1-6 and R is defined as above;
—((CR₃R₄)_{q1}—NR—CO)₂₋₄—, where R₃ and R₄ independently of each other and independently for each value of q₁ can be H, —COOH, or OH, q₁ is 1-6 and R is defined as above; or
a bond
   W₁ is arylene or heteroarylene, which may be substituted with one or two groups selected from the group consisting of —COOH, —SO₃H, and —PO₃H₂ and tetrazolyl, or W₁ is a bond;
   m is 0, 1, 2, 3, 4, 5 or 6;

### X₅ is

—O—;

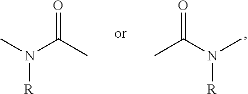

where R is defined as above; or
a bond;

### Y₁ is

—(CR₁R₂)_{q}—NR—CO—, where R₁ and R₂ independently of each other and independently for each value of q can be H, —COOH, a bond or OH, q is 1-6; and R is defined as above;
NR where R is defined as above;
—((CR₃R₄)_{q1}—NR—CO)₂₋₄—, where R₃ and R₄ independently of each other and independently for each value of q₁ can be H, —COOH, or OH, q₁ is 1-6 and R is defined as above; or
a bond;

### Q₇ is

—(CH₂)ᵣ— where r is an integer from 4 to 22;
a divalent hydrocarbon chain comprising 1, 2 or 3 —CH═CH— groups and a number of —CH₂— groups sufficient to give a total number of carbon atoms in the chain in the range of 4 to 22; or
a divalent hydrocarbon chain of the formula

—(CH₂)ₛ-Q₈-(C₆H₄)ᵥ₁-Q₉-(CH₂)_{W}-Q₁₀-(C₆H₄)ᵥ₂-Q₁₁-(CH₂)ₜ-Q₁₂-(C₆H₄)ᵥ₃-Q₁₃-(CH₂)_{z}—

wherein Q₈-Q₁₃ independently of each other can be O; S or a bond; where s, w, t and z independently of each other are zero or an integer from 1 to 10 so that the sum of s, w, t and z is in the range from 4 to 22, and v₁, v₂, and v₃ independently of each other can be zero or 1,
provided that when W₁ is a bond then Q₇ is not a divalent hydrocarbon chain of the formula (CH₂)ᵥ₄C₆H₄(CH₂)_{W1}— wherein v₄ and w₁ are integers or one of them is zero so that the sum of v₄ and w₁ is in the range of 6 to 22; and

### Z₁ is:

—COOH;
—CO-Asp;
—CO-Glu;
—CO-Gly;
—CO-Sar;
—CH(COOH)₂;
—N(CH₂COOH)₂;
—SO₃H
—PO₃H₂;
—O—SO₃H;
—O—PO₃H₂;
-tetrazolyl or
O—W₂,
where W₂ is arylene or heteroarylene substituted with one or two groups selected from —COOH, —SO₃H, and —PO₃H₂ and tetrazolyl;
provided that if W₁ is a bond and v₁, v₂ and v₃ are all zero and Q₈₋₁₃ are all a bonds, then Z₁ is O—W₂
   and any Zn²⁺ complex thereof.
14. The insulin preparation according to any one of embodiments 1 or 13, wherein W₁ is phenylene.
15. The insulin preparation according to any one of embodiments 1 or 13, wherein W₁ is 5-7 membered heterocyclic ring system comprising nitrogen, oxygen or sulphur.
16. The insulin preparation according to any one of embodiments 1, 13 and 15, wherein W₁ is a 5 membered heterocyclic ring system comprising at least one oxygen.
17. The insulin preparation according to any one of embodiments 13-16, wherein Q₇ is —(CH₂)ᵣ— where r is an integer in the range of from 4 to 22, from 8- to 20, from 12 to 20 or from 14-18.
18. The insulin preparation according to any one of the previous embodiments 13-16, wherein Q₈, Q₉, Q₁₂ and Q₁₃ are all bonds, v₂ is 1 and v₁ and v₃ are zero.
19. The insulin preparation according to any embodiment 18, wherein Q₁₀ and Q₁₁ are oxygen.
20. The insulin preparation according to any one of embodiments 13-19, wherein X₄ and Y₁ are a bonds and X₅ is

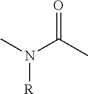

where R is —(CH₂)ₚ—COOH, where p is 1-4.
21. The insulin preparation according to any one of embodiments 13-20, wherein Z₁ is COOH.
22. The insulin preparation according to any one of embodiments 1-7 and 9, wherein the acylated insulin or analogue thereof is having a formula

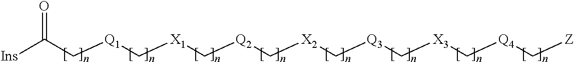

wherein Ins is the parent insulin moiety which via the α-amino group of the N-terminal amino acid residue of the B chain or an ε-amino group of a Lys residue present in the B chain of the insulin moiety is bound to the CO— group in the side chain via an amide bond;
each n is independently 0, 1, 2, 3, 4, 5 or 6;
Q₁, Q₂, Q₃, and Q₄ independently of each other can be
(CH₂CH₂O)ₛ—; (CH₂CH₂CH₂O)ₛ—; (CH₂CH₂CH₂CH₂O)ₛ—; (CH₂CH₂OCH₂CH₂CH₂CH₂O)ₛ— or (CH₂CH₂CH₂OCH₂CH₂CH₂CH₂O)ₛ— where s is 1-20
—(CH₂)ᵣ— where r is an integer from 4 to 22; or a divalent hydrocarbon chain comprising 1, 2 or 3 —CH═CH— groups and a number of —CH₂— groups sufficient to give a total number of carbon atoms in the chain in the range of 4 to 22;
—(CH₂)ₜ— or —(CH₂OCH₂)ₜ—, where t is an integer from 1 to 6;
—(CR₁R₂)_{q}—, where R₁ and R₂ independently of each other can be H, —COOH, (CH₂)₁₋₆COOH and R₁ and R₂ can be different at each carbon, and q is 1-6,
—((CR₃R₄)_{q1})₁—(NHCO—(CR₃R₄)_{q1}—NHCO)₁₋₂—((CR₃R₄)_{q1})₁ or —((CR₃R₄)_{q1})₁—(CONH—(CR₃R₄)_{q1}—CONH)₁₋₂—((CR₃R₄)_{q1}—)—, —((CR₃R₄)_{q1})₁—(NHCO—(CR₃R₄)_{q1}—CONH)₁₋₂—((CR₃R₄)_{q1})₁ or —((CR₃R₄)_{q1})₁—(CONH—(CR₃R₄)_{q1}—NHCO)₁₋₂—((CR₃R₄)_{q1})₁ where R₃ and R₄ independently of each other can be H, —COOH, and R₃ and R₄ can be different at each carbon, and q₁ is 1-6-, or
a bond;

with the proviso that Q₁-Q₄ are different;

X₁, X₂ and X₃ are independently
O;
a bond; or

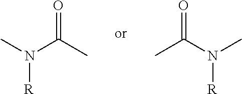

where R is hydrogen or —(CH₂)ₚ—COOH, —(CH₂)ₚ—SO₃H, —(CH₂)ₚ—PO₃H₂, —(CH₂)ₚ—O—SO₃H; —(CH₂)ₚ—O—PO₃H₂; or —(CH₂)ₚ-tetrazol-5-yl, where each p independently of the other p's is an integer in the range of 1 to 6; and

Z is:

—COOH;

—CO-Asp;

—CO-Glu;

—CO-Gly;

—CO-Sar;

—CH(COOH)₂,

—N(CH₂COOH)₂;

—SO₃H

—OSO₃H
—OPO3H₂

—PO₃H₂ or

-tetrazol-5-yl

and any Zn²⁺ complex thereof.
23. The insulin preparation according to any one of embodiments 1 or 22, wherein s is in the range of 2-12, 2-4 or 2-3.
24. The insulin preparation according to any one of embodiments 1 or 22, wherein s is preferably 1.
25. The insulin preparation according to any one of embodiments 22-24, wherein Z is —COOH.
26. The insulin preparation according to any one of embodiments, wherein the parent insulin is a desB30 human insulin analogue.
27. The insulin preparation according to any of the previous embodiments, wherein the parent insulin is selected from the group consisting of human insulin; desB1 human insulin; desB30 human insulin; GlyA21 human insulin; GlyA21 desB30 human insulin; AspB28 human insulin; porcine insulin; LysB28 ProB29 human insulin; and LysB3 GluB29 human insulin or AspB28 desB30 human insulin.
28. The insulin preparation according to any one of the previous embodiments 1-8, 13-21 and 26-27, wherein the acylated insulin or analog thereof is selected from the group consisting of 0100-0000-0496 N^{εB29}—[N—(HOOC(CH₂)₁₄CO)—N-(carboxyethyl)-CH₂—C₆H₄CO] desB30 human insulin; 0100-0000-0515 N^{εB29}—[N—(HOOC(CH₂)₁₃CO)—N-(carboxyethyl)CH₂—C₆H₄CO] desB30 human insulin; 0100-0000-0522 N^{εB29}—[N—(HOOC(CH₂)₁₅CO)—N-(carboxyethyl)-CH₂—C₆H₄CO] desB30 human insulin; 0100-0000-0488 N^{εB29}—[N—(HOOC(CH₂)₁₆CO)—N-(carboxyethyl)-CH₂—C₆H₄CO] desB30 human insulin; 0100-0000-0544 N^{εB29}—[N—(HOOC(CH₂)₁₄CO)—N-(carboxymethyl)-C₆H₄CO] desB30 human insulin, and 0100-0000-029 N^{εB29}—[N—(HOOC(CH₂)₁₄CO)—N-(carboxyethyl)-CH₂— (furanylene)CO] desB30 human insulin, 0100-0000-0552 N^{εB29}-{4-Carboxy-4-[10-(4-carboxy-phenoxy)decanoylamino]-butyryl}desB30 human insulin.
29. The insulin preparation according to any of the previous embodiments, wherein the acylated insulin or analog thereof is selected from the group consisting of N^{εB29}-(3-[2-{2-(2-[ω-carboxy-pentadecanoyl-γ-glutamyl-(2-amino-ethoxy)]-ethoxy)-ethoxy}-ethoxy]-propinoyl) desB30 human insulin, N^{εB29}-(3-[2-{2-(2-[ω-carboxy-heptadecanoyl-γ-glutamyl-(2-amino-ethoxy)]-ethoxy)-ethoxy}-ethoxy]-propinoyl) desB30 human insulin, N^{εB29}-{3-[2-(2-{2-[2-(ω-carboxy-pentadecanoylamino)-ethoxy]-ethoxy}-ethoxy)-ethoxy]-propionyl-γ-glutamyl desB30 human insulin, N^{εB29}-(ω-[2-(2-{2-[2-(2-carboxy-ethoxy)-ethoxy]-ethoxy}-ethoxy)-ethylcarbamoyl]-heptadecanoyl-α-glutamyl) desB30 human insulin, N^{εB29}-(ω-[2-(2-{2-[2-(2-carboxy-ethoxy)-ethoxy]-ethoxy}-ethoxy)-ethylcarbamoyl]-heptadecanoyl-γ-glutamyl) desB30 human insulin, N^{εB29}-3-[2-(2-{2-[2-(ω-carboxy-heptadecanoylamino)-ethoxy]-ethoxy}-ethoxy)-ethoxy]-propionyl-γ-glutamyl desB30 human insulin, N^{εB29}-(3-(3-{2-[2-(3-[7-carboxyheptanoylamino]propoxy)ethoxy}-ethoxy]propylcarbamoyl)propionyl) desB30 human insulin, N^{εB29}-(3-(3-{4-[3-(7-Carboxyheptanoylamino)propoxy]butoxy}propylcarbamoyl)-propionyl-γ-glutamyl) desB30 human insulin, N^{εB29}-(3-(3-{2-[2-(3-[9-Carboxynonanoylamino]propoxy)ethoxy]ethoxy}-propylcarbamoyl)propionyl) desB30 human insulin, N^{εB29}-(3-(2-{2-[2-(9-carboxynonanoylamino)ethoxy]ethoxy}ethylcarbamoyl)propionyl-γ-glutamyl) desB30 human insulin, N^{εB29}-(3-(3-{4-[3-(9-Carboxynonanoylamino)propoxy]butoxy}-propylcarbamoyl)propionyl-γ-glutamyl) desB30 human insulin, N^{εB29}-(2-[3-(2-(2-{2-(7-carboxyheptanoylamino)ethoxy}ethoxy)-ethylcarbamoyl]propionyl-γ-glutamyl) desB30 human insulin, N^{εB29}-(3-[2-(2-{2-[2-(ω-carboxypentadecanoylamino)ethoxy]ethoxy}-ethoxy)ethoxy]propionyl)) desB30 human insulin, N^{εB29}-(3-(2-{2-[2-(2-{2-[2-(2-{2-[2-(2-{2-[2-(ω-carboxy-tridecanoylamino)-ethoxy]-ethoxy}-ethoxy)-ethoxy]-ethoxy}-ethoxy)ethoxy]-ethoxy}-ethoxy)-ethoxy]-ethoxy}-ethoxy)-propionoyl-γ-glutamyl) desB30 human insulin, N^{εB29}-(3-[2-(2-{2-[2-(ω-Carboxy-tridecanoylamino)-ethoxy]-ethoxy}-ethoxy)ethoxy]-propionoyl-γ-glutamyl) desB30 human insulin, N^{εB29}-(3-[2-(2-{2-[2-(2-{2-[2-(ω-carboxy-tridecanoylamino)-ethoxy]ethoxy}-ethoxy)-ethoxy]-ethoxy}-ethoxy)-ethoxy]-ethoxy}-propionoyl-γ-glutamyl) desB30 human insulin, N^{εB29}-(3-(2-{2-[2-(ω)-Carboxy-pentadecanoylamino)-ethoxy]-ethoxy}-ethylcarbamoyl)-propionyl-γ-glutamyl) desB30 human insulin, N^{εB29}-(3-(3-{2-[2-(3-[ω-Carboxypentadecanoylamino]propoxy)ethoxy]-ethoxy}propylcarbamoyl)propionyl-γ-glutamyl) desB30 human insulin, N^{εB29}-(3-(3-{4-[3-(ω-Carboxyundecanoylamino)propoxy]butoxypropylcarbamoyl)propionyl-γ-glutamyl) desB30 human insulin, N^{εB29}-(3-(3-{4-[3-(ω-carboxytridecanoylamino)propoxy]butoxypropylcarbamoyl)propionyl-γ-glutamyl) desB30 human insulin, N^{εB29}-(3-(2-{2-[2-(ω-Carboxyundecanoylamino)ethoxy]ethoxy}ethylcarbamoyl)propionyl-γ-glutamyl) desB30 human insulin, N^{εB29}-(3-(2-{2-[2-(ω-carboxytridecanoylamino)ethoxy]ethoxy}-ethylcarbamoyl)propionyl-γ-glutamyl) desB30 human insulin, N^{εB29}-{3-[2-(2-{2-[2-(ω-carboxy-pentadecanoylamino)ethoxy]ethoxy}ethoxy)ethoxy]propionyl-gamma-γ-D-glutamyl) desB30 human insulin, N^{εB29}-{3-[2-(2-{2-[2-(7-carboxyheptanoylamino)ethoxy]-ethoxy}ethoxy)ethoxy]propionyl-γ-glutamyl} desB30 human insulin, N^{εB29}-{3-[2-(2-{2-[2-(9-carboxynonanoylamino)ethoxy]ethoxy}ethoxy)ethoxy]propionyl-γ-glutamyl} desB30 human insulin, N^{εB29}-{3-[2-(2-{2-[2-(ω-carboxyundecanoylamino)ethoxy]ethoxy}ethoxy)-ethoxy]propionyl-γ-glutamyl} desB30 human insulin, N^{B29}-{3-[2-(2-{2-[2-(ω-carboxytridecanoylamino)ethoxy]ethoxy}ethoxy)ethoxy]propionyl-γ-glutamyl} desB30 human insulin.
30. The insulin preparation according to any one of the previous embodiments, wherein the acylated insulin or analog thereof is NεB29-hexadecandiyol-γ-Glu-(desB30) human insulin.
31. The insulin preparation according to any one of the previous embodiments, wherein the acylated insulin or analog thereof is insulin detemir (N^{εB29}-myristoyl) desB30 human insulin).
32. The insulin preparation according to any one of the previous embodiments, wherein at least 85% of the acylated insulin or analog thereof is present as complexes which are acylated insulin dodecamers or complexes with a higher molar weight than acylated insulin dodecamer.
33. The insulin preparation according to any one of the previous embodiments, wherein at least 92% of the acylated insulin or analog thereof is present as complexes which are acylated insulin dodecamers or complexes with a higher molar weight than acylated insulin dodecamer.
34. The insulin preparation according to any one of the previous embodiments, wherein at least 95% of the acylated insulin or analog thereof is present as complexes which are acylated insulin dodecamers or complexes with a higher molar weight than acylated insulin dodecamer.
35. The insulin preparation according to any one of the previous embodiments, wherein at least 97% of the acylated insulin or analog thereof is present as complexes which are acylated insulin dodecamers or complexes with a higher molar weight than acylated insulin dodecamer.
36. The insulin preparation according to any one of the previous embodiments, wherein the insulin is human insulin or an insulin analog.
37. The insulin preparation according to any one of the previous embodiments, wherein the insulin analog is B28Asp human insulin.
38. The insulin preparation according to any of the previous embodiments, wherein the insulin analog is B28LysB29Pro human insulin.
39. The insulin preparation according to any of the previous embodiments, wherein the insulin analog is B3LysB29Glu human insulin.
40. The insulin preparation according to any of the previous embodiments, wherein at least 85% of the human insulin or insulin analog is present as rapid acting insulin hexamer or complexes with a smaller molar weight than rapid acting insulin hexamers.
41. The insulin preparation according to any of the previous embodiments, wherein at least 92% of the human insulin or insulin analog is present as rapid acting insulin hexamer or complexes with a smaller molar weight than rapid acting insulin hexamers.
42. The insulin preparation according to any of the previous embodiments, wherein at least 95% of the human insulin or insulin analog is present as rapid acting insulin hexamer or complexes with a smaller molar weight than rapid acting insulin hexamers.
43. The insulin preparation according to any of the previous embodiments, wherein at least 97% of the human insulin or insulin analog is present as rapid acting insulin hexamer or complexes with a smaller molar weight than rapid acting insulin hexamers.
44. The insulin preparation according to any of the previous embodiments, wherein at least 99% of the human insulin or insulin analog is present as rapid acting insulin hexamer or complexes with a smaller molar weight than rapid acting insulin hexamers.
45. The insulin preparation according to any of the preceding embodiments, wherein the acylated insulin, analogues thereof and human insulin or analogues thereof are present in a range selected from the following: 0.1-10.0 mM; 0.1-3.0 mM; 0.1-2.5 mM; 0.1-2.0 mM; 0.1-1.5 mM; 0.2-2.5 mM; 0.2-2.0 mM; 0.2-1.5 mM; 0.3-3.0 mM; 0.3-2.5 mM; 0.3-2.0 mM; 0.3-1.5 mM; 0.5-1.3 mM and 0.6-1.2 mM.
46. The insulin preparation according to any of the preceding embodiments, wherein the acylated insulin, analogues thereof and human insulin or analogues thereof are present in the amount from about 0.1 mM to about 10.0 mM.
47. The insulin preparation according to any of the preceding embodiments, wherein the acylated insulin, analogues thereof and human insulin or analogues thereof are present in the amount from about 0.1 mM to about 3.0 mM.
48. The insulin preparation according to any of the preceding embodiments, wherein the acylated insulin, analogues thereof and human insulin or analogues thereof are present in the amount from about 0.1 mM to about 2.5 mM.
49. The insulin preparation according to any of the preceding embodiments, wherein the acylated insulin, analogues thereof and human insulin or analogues thereof are present in the amount from about 0.1 mM to about 2.0 mM.
50. The insulin preparation according to any of the preceding embodiments, wherein the long-acting and fast-acting insulin compounds are present in the amount from about 0.1 mM to about 1.5 mM.
51. The insulin preparation according to any of the preceding embodiments, wherein the acylated insulin, analogues thereof and human insulin or analogues thereof are present in the amount from about 0.2 mM to about 2.5 mM.
52. The insulin preparation according to any of the preceding embodiments, wherein the acylated insulin, analogues thereof and human insulin or analogues thereof are present in the amount from about 0.2 mM to about 2.0 mM.
53. The insulin preparation according to any of the preceding embodiments, wherein the acylated insulin, analogues thereof and human insulin or analogues thereof are present in the amount from about 0.2 mM to about 1.5 mM.
54. The insulin preparation according to any of the preceding embodiments, wherein the acylated insulin, analogues thereof and human insulin or analogues thereof are present in the amount from about 0.3 mM to about 3.0 mM.
55. The insulin preparation according to any of the preceding embodiments, wherein the acylated insulin, analogues thereof and human insulin or analogues thereof are present in the amount from about 0.3 mM to about 2.5 mM.
56. The insulin preparation according to any of the preceding embodiments, wherein the acylated insulin, analogues thereof and human insulin or analogues thereof are present in the amount from about 0.1 mM to about 2.0 mM.
57. The insulin preparation according to any of the preceding embodiments, wherein the acylated insulin, analogues thereof and human insulin or analogues thereof are present in the amount from about 0.1 mM to about 1.5 mM.
58. The insulin preparation according to any of the preceding embodiments, wherein the acylated insulin, analogues thereof and human insulin or analogues thereof are present in the amount from about 0.15 mM to about 1.3 mM.
59. The insulin preparation according to any of the preceding embodiments, wherein the acylated insulin, analogues thereof and human insulin or analogues thereof are present in the amount from about 0.15 mM to about 1.2 mM.
60. The insulin preparation according to any of the preceding embodiments, wherein the acylated insulin, analogues thereof and human insulin or analogues thereof are present in the amount from about 0.15 mM to about 1.2 mM.
61. The insulin preparation according to any of the preceding embodiments, wherein the acylated insulin, analogues thereof and human insulin or analogues thereof are present in the amount from about 0.15 mM to about 0.5 mM
62. The insulin preparation according to any of the preceding embodiments, wherein the acylated insulin, analogues thereof and human insulin or analog thereof are present in the amount of about 0.3 mM.
63. The insulin preparation according to any of the preceding embodiments, wherein the acylated insulin, analogues thereof and human insulin or analog thereof are present in the amount of about 0.6M.
64. The insulin preparation according to any of the preceding embodiments, wherein the acylated insulin or an analog thereof is present in the amount of about 0.42 mM and the fast acting insulin compound is present in the amount of about 0.18 mM.
65. The insulin preparation according to any of the preceding embodiments, wherein the acylated insulin or an analog thereof is present in the amount of about 0.18 mM and the human insulin or analog thereof is present in the amount of about 0.42 mM.
66. The insulin preparation according to any of the preceding embodiments, wherein the acylated insulin or an analog thereof is present in the amount of about 0.84 mM and the human insulin or analog thereof is present in the amount of about 0.36 mM.
67. The insulin preparation according to any of the preceding embodiments, wherein the acylated insulin or an analog thereof is present in the amount of about 0.36 mM and the human insulin or analog thereof is present in the amount of about 0.84 mM.
68. The insulin preparation according to any of the preceding embodiments, wherein the sum of acylated insulin or an analog thereof and human insulin or analog thereof is present in the amount of about 0.6 mM
69. The insulin preparation according to any of the preceding embodiments, wherein the sum of acylated insulin or an analog thereof and human insulin or analog thereof is present in the amount of about 1.2 mM.
70. The insulin preparation according to any of the preceding embodiments, wherein the acylated insulin or an analog thereof is present in 70% and the human insulin or analog thereof is present in about 30%.
71. The insulin preparation according to any of the preceding embodiments, wherein the nicotinic compound is selected from the group consisting of nicotinamide, nicotinic acid, niacin, niacin amide and vitamin B3 and/or salts thereof and/or any combination thereof.
72. The insulin preparation according to any of the preceding embodiments, wherein the nicotinic compound is selected from nicotinamide and nicotinic acid and/or salts thereof and/or any combination thereof.
73. The insulin preparation according to any of the preceding embodiments, wherein the nicotinic compound is nicotinamide and/or salts thereof.
74. The insulin preparation according to any of the preceding embodiments, wherein the nicotinic compound is present in a range selected from the following: 1-300 mM; 5-200 mM; 10-150 mM, 20-140 mM or 20-100 mM.
75. The insulin preparation according to any of the preceding embodiments, comprising from about 1 mM to about 300 mM of the nicotinic compound.
76. The insulin preparation according to any of the preceding embodiments, comprising from about 8 mM to about 260 mM of the nicotinic compound.
77. The insulin preparation according to any of the preceding embodiments, comprising from about 10 mM to about 200 mM of the nicotinic compound.
78. The insulin preparation according to any of the preceding embodiments, comprising from about 10 mM to about 150 mM of the nicotinic compound.
79. The insulin preparation according to any of the preceding embodiments, comprising from about 5 mM to about 20 mM of the nicotinic compound.
80. The insulin preparation according to any of the preceding embodiments, comprising from about 20 mM to about 120 mM of the nicotinic compound.
81. The insulin preparation according to any of the preceding embodiments, comprising from about 40 mM to about 120 mM of the nicotinic compound.
82. The insulin preparation according to any of the preceding embodiments, comprising from about 20 mM to about 40 mM of the nicotinic compound.
83. The insulin preparation according to any of the preceding embodiments, comprising from about 40 mM to about 80 mM of the nicotinic compound.
84. The insulin preparation according to any of the preceding embodiments, comprising from about 20 mM to about 100 mM of the nicotinic compound.
85. The insulin preparation according to any of the preceding embodiments, comprising from about 30 mM to about 130 mM of the nicotinic compound.
86. The insulin preparation according to any of the preceding embodiments, comprising about 8 mM, 20 mM, 40 mM, 100 mM or 120 mM of the nicotinic compound.
87. The insulin preparation according to any of the preceding embodiments, comprising about 8 mM of the nicotinic compound.
88. The insulin preparation according to any of the preceding embodiments, comprising about 30 mM, 70 mM, 100 mM or 130 mM of the nicotinic compound.
89. The insulin preparation according to any of the preceding embodiments, comprising about 40 mM of the nicotinic compound.
90. The insulin preparation according to any of the preceding embodiments, comprising about 80 mM of the nicotinic compound.
91. The insulin preparation according to any of the preceding embodiments, comprising about 120 mM of the nicotinic compound.
92. The insulin preparation according to any of the preceding embodiments, comprising about 150 mM of the nicotinic compound.
93. The insulin preparation according to any of the preceding embodiments, comprising the following ranges of arginine compound: 1-100 mM, 5-120 mM, 8-50 mM, 5-50 mM, 5-30 mM, 8-30 mM, 10-30 mM, 30-60 mM or 10-40 mM.
94. The insulin preparation according to any of the preceding embodiments, comprising the following ranges of arginine compound: 1-120 mM, 8-85 mM or 1-40 mM.
95. The insulin preparation according to any of the preceding embodiments, comprising from about 1 mM to about 120 mM of arginine.
96. The insulin preparation according to any of the preceding embodiments, comprising from about 1 mM to about 100 mM of arginine.
97. The insulin preparation according to any of the preceding embodiments, comprising from about 5 mM to about 80 mM of arginine.
98. The insulin preparation according to any of the preceding embodiments, comprising from about 20 mM to about 80 mM of arginine.
99. The insulin preparation according to any of the preceding embodiments, comprising from about 5 mM to about 25 mM of arginine.
100. The insulin preparation according to any of the preceding embodiments, comprising from about 8 mM to about 85 mM of arginine.
101. The insulin preparation according to any of the preceding embodiments, comprising from about 10 mM to about 60 mM of arginine.
102. The insulin preparation according to any of the preceding embodiments, comprising from about 10 mM to about 40 mM of arginine.
103. The insulin preparation according to any of the preceding embodiments, comprising from about 1 mM to about 40 mM of arginine.
104. The insulin preparation according to any of the preceding embodiments, wherein arginine is present in a range selected from the following: 1 mM, 2 mM, 3 mM, 4 mM, 5 mM, 6 mM, 7 mM, 8 mM, 9 mM, 10 mM, 12 mM, 15 mM, 20 mM, 25 mM, 30 mM, 35 mM or 40 mM, 45 mM, 50 mM, 55 mM or 60 mM.
105. The insulin preparation according to any of the preceding embodiments, comprising about 1 mM of arginine.
106. The insulin preparation according to any of the preceding embodiments, comprising about 2 mM of arginine.
107. The insulin preparation according to any of the preceding embodiments, comprising about 3 mM of arginine.
108. The insulin preparation according to any of the preceding embodiments, comprising about 4 mM of arginine.
109. The insulin preparation according to any of the preceding embodiments, comprising about 5 mM of arginine.
110. The insulin preparation according to any of the preceding embodiments, comprising about 6 mM of arginine.
111. The insulin preparation according to any of the preceding embodiments, comprising about 7 mM of arginine.
112. The insulin preparation according to any of the preceding embodiments, comprising about 8 mM of arginine.
113. The insulin preparation according to any of the preceding embodiments, comprising about 10 mM of arginine.
114. The insulin preparation according to any of the preceding embodiments, comprising about 15 mM of arginine.
115. The insulin preparation according to any of the preceding embodiments, comprising about 20 mM of arginine.
116. The insulin preparation according to any of the preceding embodiments, comprising about 25 mM of arginine.
117. The insulin preparation according to any of the preceding embodiments, comprising about 30 mM of arginine.
118. The insulin preparation according to any of the preceding embodiments, comprising about 35 mM of arginine.
119. The insulin preparation according to any of the preceding embodiments, comprising about 40 mM of arginine.
120. The insulin preparation according to any of the preceding embodiments, comprising about 45 mM of arginine.
121. The insulin preparation according to any of the preceding embodiments, comprising about 50 mM of arginine.
122. The insulin preparation according to any of the preceding embodiments, comprising about 55 mM of arginine.
123. The insulin preparation according to any of the preceding embodiments, comprising about 60 mM of arginine.
124. The insulin preparation according to any of the preceding embodiments, which further comprises a buffer(s).
125. The insulin preparation according to embodiment 124, wherein said buffer is Tris.
126. The insulin preparation according to embodiment 125, comprising from about 2 mM to about 50 mM of Tris.
127. The insulin preparation according to embodiment 125, comprising from about 3 mM to about 40 mM of Tris.
128. The insulin preparation according to embodiment 125, comprising from about 20 mM to about 30 mM of Tris.
129. The insulin preparation according to embodiment 125, comprising about 7 mM, 10 mM, 20 mM, 30 mM or 40 mM of Tris.
130. The insulin preparation according to embodiment 125, comprising about 7 mM of Tris.
131. The insulin preparation according to embodiment 125, comprising about 10 mM of Tris.
132. The insulin preparation according to embodiment 125, comprising about 20 mM of Tris.
133. The insulin preparation according to embodiment 125, comprising about 30 mM of Tris.
134. The insulin preparation according to embodiment 125, comprising about 40 mM of Tris.
135. The insulin preparation according to any of the previous embodiments, which further comprises a metal ion.
136. The insulin preparation according to embodiment 135, wherein the metal ion is zinc.
137. The insulin preparation according to embodiment 136, wherein less than about 6 zinc ions are present per 6 insulin compounds.
138. The insulin preparation according to embodiment 136, wherein less than about 5 zinc ions are present per 6 insulin compounds.
139. The insulin preparation according to embodiment 136, wherein less than about 4.5 zinc ions are present per 6 insulin compounds.
140. The insulin preparation according to embodiment 136, wherein about 4.2 zinc ions are present per 6 insulin compounds, wherein the percentage of long-acting insulin compound is 70% and the percentage of fast-acting insulin compound is 30%.
141. The insulin preparation according to embodiment 136, wherein about 4.7 zinc ions per 6 long-acting insulin compounds is combined with about 3 zinc ions per 6 fast-acting insulin compounds.
142. The insulin preparation according to embodiment 136, wherein the zinc:insulin molar ratio is from about 2:6 to about 6:6.
143. The insulin preparation according to embodiment 136, wherein the zinc:insulin molar ratio is from about 3:6 to about 5:6.
144. The insulin preparation according to embodiment 136, wherein the zinc:insulin molar ratio for the long-acting insulin compound is from about 4:6 to about 6:6 and for the short-acting insulin compound below 4:6 before combination.
145. The insulin preparation according to embodiment 136, wherein the zinc:insulin molar ratio is about 2.5:6.
146. The insulin preparation according to embodiment 136, wherein the zinc:insulin molar ratio is about 3:6.
147. The insulin preparation according to embodiment 136, wherein the zinc:insulin molar ratio is about 3.5:6.
148. The insulin preparation according to embodiment 136, wherein the zinc:insulin molar ratio is about 4:6.
149. The insulin preparation according to embodiment 136, wherein the zinc:insulin molar ratio is about 4.5:6.
150. The insulin preparation according to embodiment 136, wherein the zinc:insulin molar ratio is about 5:6.
151. The insulin preparation according to any of the preceding embodiments, which further comprises a stabilizer(s).
152. The insulin preparation according to embodiment 151, wherein the stabilizer is a non-ionic detergent.
153. The insulin preparation according to embodiment 152, wherein the detergent is polysorbate 20 (Tween 20) or polysorbate 80 (Tween 80).
154. The insulin preparation according to embodiment 152, wherein the detergent is polysorbate 20 (Tween 20).
155. The insulin preparation according to embodiment 152, wherein the detergent is polysorbate 80 (Tween 80).
156. The insulin preparation according to any of embodiments 153-155, comprising from about 5 to 100 ppm, from about 10 to about 50 ppm or from about 10 to about 20 ppm of polysorbate.
157. The insulin preparation according to any of the preceding embodiments, which further comprises one or more preservative agent(s).
158. The insulin preparation according to embodiment 157, wherein said preservative is a phenolic compound.
159. The insulin preparation according to embodiment 158, wherein said phenolic compound is present in the amount from about 0 to about 6 mg/ml or from about 0 to about 4 mg/ml.
160. The insulin preparation according to embodiment 158, wherein said phenolic compound is present in the amount of from about 5 to about 70 mM.
161. The insulin preparation according to embodiment 158, wherein said phenolic compound is present in the amount of from about 5 to about 50 mM.
162. The insulin preparation according to embodiment 158, wherein said phenolic compound is present in the amount of from about 5 to about 30 mM.
163. The insulin preparation according to embodiment 158, wherein said phenolic compound is present in the amount of about 16 mM.
164. The insulin preparation according to embodiment 158, wherein said phenolic compound is present in the amount of about 19 mM.
165. The insulin preparation according to embodiment 157, wherein said preservative is m-cresol.
166. The insulin preparation according to embodiment 165, wherein m-cresol is present in the amount from about 0.5 to about 4.0 mg/ml.
167. The insulin preparation according to embodiment 165, wherein m-cresol is present in the amount of from about 5 to about 70 mM.
168. The insulin preparation according to embodiment 165, wherein m-cresol is present in the amount of from about 5 to about 50 mM.
169. The insulin preparation according to embodiment 165, wherein m-cresol is present in the amount of from about 5 to about 30 mM.
170. The insulin preparation according to embodiment 165, wherein m-cresol is present in the amount of about 16 mM.
171. The insulin preparation according to embodiment 165, wherein m-cresol is present in the amount of about 19 mM.
172. The insulin preparation according to any of the preceding embodiments, further comprising glycerol in the amount from about 0.5 to about 2.5%.
173. The insulin preparation according to any of the preceding embodiments, further comprising glycerol in the amount from about 0.7 to about 2.0%.
174. The insulin preparation according to any of the preceding embodiments, further comprising glycerol in the amount from about 0.8 to about 1.6%.
175. The insulin preparation according to any of the preceding embodiments, further comprising glycerol in the amount of about 1.1%.
176. An insulin preparation according to any of the previous embodiments, wherein the pH is neutral to weakly basic.
177. An insulin preparation according to any of the previous embodiments, wherein the pH is from about 7.0 to about 8.0.
178. An insulin preparation according to any of the previous embodiments, wherein the pH is about 7.0.
179. An insulin preparation according to any of the previous embodiments, wherein the pH is about 7.1.
180. An insulin preparation according to any of the previous embodiments, wherein the pH is about 7.2.
181. An insulin preparation according to any of the previous embodiments, wherein the pH is about 7.3.
182. An insulin preparation according to any of the previous embodiments, wherein the pH is about 7.4.
183. An insulin preparation according to any of the previous embodiments, wherein the pH is about 7.5.
184. An insulin preparation according to any of the previous embodiments, wherein the pH is about 7.6.
185. An insulin preparation according to any of the previous embodiments, wherein the pH is about 7.7.
186. An insulin preparation according to any of the previous embodiments, wherein the pH is about 7.8.
187. An insulin preparation according to any of the previous embodiments, wherein the pH is about 7.9.
188. An insulin preparation according to any of the previous embodiments, wherein the pH is about 8.0.
189. A Method for producing a pharmaceutical composition comprising an acylated insulin and a fast-acting insulin, wherein more than about 4 zinc atoms per 6 molecules of each of the compounds are added to the composition.
190. Method according to embodiment 189, wherein up to about 12 zinc atoms per 6 molecules of each of the insulin compounds are added to the composition.
191. Method according to any of embodiments 189-190, wherein between about 4.3 and about 12 zinc atoms per 6 molecules of each of the insulin compounds are added to the composition.
192. Method according to any of embodiments 189-191, wherein the zinc is added to the composition before addition of a preservative.
193. Method according to any of embodiments 189-192, wherein the number of zinc atoms added before addition of a preservative is more than 1 zinc atom per 6 molecules of acylated insulin.
194. Method according to any of embodiments 189-193, wherein the number of zinc atoms added before addition of a preservative is more than 2 zinc atom per 6 molecules of acylated insulin.
195. Method according to any of embodiments 189-194, wherein the number of zinc atoms added before addition of a preservative is more than 3 zinc atom per 6 molecules of acylated insulin.
196. Method according to any of embodiments 189-195, wherein the number of zinc atoms added before addition of a preservative is more than 4 zinc atom per 6 molecules of acylated insulin.
197. Method according to any of embodiments 189-196, wherein the zinc is added to the composition after addition of a preservative.
198. Method according to any of embodiments 189-197, wherein at least 0.5 zinc atom per 6 molecules of acylated insulin is added to the composition after addition of a preservative.
199. Method according to any of embodiments 189-198, wherein at least 1 zinc atom per 6 molecules of acylated insulin is added to the composition after addition of a preservative.
200. Method according to any of embodiments 189-199, wherein part of the zinc is added before addition of a preservative and part of the zinc is added after addition of a preservative.
201. Method according to any of embodiments 189-200, wherein the preservative is phenol and/or m-cresol.
202. Method according to any of embodiments 189-200, wherein up to about 14 zinc atoms per 6 molecules of acylated insulin or analog thereof are added to the composition.
203. Method according to any of embodiments 189-200, wherein between about 4.3 and about 14 zinc atoms per 6 molecules of acylated insulin or analog thereof are added to the composition.
204. Method according to any of embodiments 189-200, wherein the zinc is added to the composition before addition of a preservative.
205. Method according to any of embodiments 189-200, wherein the number of zinc atoms added before addition of a preservative is more than 1 zinc atom per 6 molecules of acylated insulin or analog thereof.
206. Method according to any of embodiments 189-200, wherein the number of zinc atoms added before addition of a preservative is more than 2 zinc atoms per 6 molecules of acylated insulin or analog thereof.
207. Method according to any of embodiments 189-200, wherein the number of zinc atoms added before addition of a preservative is more than 4 zinc atoms per 6 molecules of acylated insulin or analog thereof.
208. Method according to any of embodiments 189-200, wherein the number of zinc atoms added before addition of a preservative is more than 5 zinc atoms per 6 molecules of acylated insulin or analog thereof.
209. Method according to any of embodiments 189-200, wherein the zinc is added to the composition after addition of a preservative.
210. Method according to embodiment 209, wherein at least 0.2 zinc atom per 6 molecules of acylated insulin or analog thereof is added to the composition after addition of a preservative.
211. Method according to embodiment 209, wherein at least 1 zinc atom per 6 molecules of acylated insulin or analog thereof is added to the composition after addition of a preservative.

In a further aspect of the invention more than about 2 zinc atoms per 6 molecules of acylated insulin are added to the composition after the addition of a preservative or more than about 3 zinc atoms per 6 molecules of acylated insulin are added to the composition after the addition of a preservative or more than about 4 zinc atoms per 6 molecules of acylated insulin are added to the composition after the addition of a preservative.

In a further aspect of the invention between about 4.5 and about 12 zinc atoms per 6 molecules of acylated insulin are added to the composition after the addition of a preservative or more preferred about 5 and about 11.4 zinc atoms per 6 molecules of acylated insulin are added to the composition after the addition of a preservative or even more preferred between about 5.5 and about 10 zinc atoms per 6 molecules of acylated insulin are added to the composition after the addition of a preservative.
212. Method according to any of the previous embodiments, wherein part of the zinc is added before addition of a preservative and part of the zinc is added after addition of a preservative.

In one embodiment the method comprises adding at least 1 zinc atom per 6 molecules of acylated insulin before addition of a preservative and adding at least 1 zinc atom per 6 molecules of acylated insulin after addition of a preservative or adding at least 1 zinc atom per 6 molecules of acylated insulin before addition of a preservative and adding at least 2-3 zinc atoms per 6 molecules of acylated insulin after addition of a preservative or adding at least 1 zinc atom per 6 molecules of acylated insulin before addition of a preservative and up to about 11 zinc atom per 6 molecules of acylated insulin and after addition of a preservative.

In one embodiment the method comprises adding at least 2 zinc atoms per 6 molecules of acylated insulin before addition of a preservative and adding at least 1 zinc atom per 6 molecules of acylated insulin after addition of a preservative or adding at least 2 zinc atoms per 6 molecules of acylated insulin before addition of a preservative and adding at least 2-3 zinc atoms per 6 molecules of acylated insulin after addition of a preservative or adding at least 2 zinc atoms per 6 molecules of acylated insulin before addition of a preservative and up to about 10 zinc atoms per 6 molecules of acylated insulin after addition of a preservative.

In one embodiment the method comprises adding at least 3 zinc atoms per 6 molecules of acylated insulin before addition of a preservative and adding at least 1 zinc atom per 6 molecules of acylated insulin after addition of a preservative or adding at least 3 zinc atoms per 6 molecules of acylated insulin before addition of a preservative and adding at least 2-3 zinc atoms per 6 molecules of acylated insulin after addition of a preservative or adding at least 3 zinc atoms per 6 molecules of acylated insulin before addition of a preservative and up to about 9 zinc atoms per 6 molecules of acylated insulin after addition of a preservative.
213. Method according to embodiment 212, wherein the preservative is phenol and/or m-cresol.
214. A method of reducing the blood glucose level in mammals by administering to a patient in need of such treatment a therapeutically active dose of an insulin preparation according to any of the previous embodiments.
215. A method for the treatment of diabetes mellitus in a subject comprising administering to a subject an insulin preparation according to any of the previous embodiments.
216. A method according to any of the previous embodiments, for parenteral administration.
217. An insulin preparation according to any one of embodiments 1-188, for use in the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, stroke, coronary heart disease and other cardiovascular disorders and treatment of critically ill diabetic and non-diabetic patients.
218. An insulin preparation according to any one of embodiments 1-188, for use in the treatment or prevention of hyperglycemia including stress induced hyperglycemia, type 2 diabetes, impaired glucose tolerance, type 1 diabetes, and burns, operation wounds and other diseases, myocardial infarction, stroke, coronary heart disease and other cardiovascular disorders.

Further embodiments of the invention relate to the following:
219. An insulin preparation comprising:
   a long-acting insulin compound,
   a fast-acting insulin compound,
   a nicotinic compound, and
   arginine.
220. The insulin preparation according to embodiment 219, wherein the long-acting insulin is an acylated insulin.
221. The insulin preparation according to embodiments 219-220, wherein the acylated insulin is an insulin acylated in the ε-amino group of a Lys residue in a position in the B-chain of the parent insulin molecule.
222. The insulin preparation according to embodiment 221, wherein the parent insulin is selected from the group consisting of human insulin; desB1 human insulin; desB30 human insulin; GlyA21 human insulin; GlyA21 desB30 human insulin; AspB28 human insulin; porcine insulin; LysB28 ProB29 human insulin; GlyA21 ArgB31 ArgB32 human insulin; and LysB3 GluB29 human insulin or AspB28 desB30 human insulin.
223. The insulin preparation according to embodiment 222, wherein the acylated insulin is Nε1329-hexadecandiyol-γ-Glu-(desB30) human insulin.
224. The insulin preparation according to embodiment 222, wherein the long-acting insulin is selected from the group consisting of N^{εB29}-myristoyl (desB30) human insulin and A21GlyB31ArgB32Arg human insulin.
225. The insulin preparation according to any one of embodiments 219-224, wherein the fast-acting insulin is human insulin or an insulin analog.
226. The insulin preparation according to any one of embodiments 219-225, wherein the fast-acting insulin is B28Asp human insulin.
227. The insulin preparation according to any one of embodiments 219-226, wherein the fast-acting insulin compound is selected from the group consisting of B28LysB29Pro human insulin and B3LysB29Glu human insulin.
228. The insulin preparation according to any one of embodiments 219-227, wherein the long-acting and fast-acting insulin compounds are present in the amount from about 0.1 mM to about 10.0 mM.
229. The insulin preparation according to any one of embodiments 219-228, wherein the long-acting insulin compound is present in about 70% and the fast-acting insulin compounds is present in about 30%.
230. The insulin preparation according to any one of embodiments 219-229, wherein the nicotinic compound is selected from the group consisting of nicotinamide, nicotinic acid, niacin, niacin amide and vitamin B3 and/or salts thereof and/or any combination thereof.
231. The insulin preparation according to any one of embodiments 219-230, comprising from about 1 mM to about 120 mM of arginine.
232. The insulin preparation according to any one of embodiments 219-231, which further comprises a buffer(s) and/or a metal ion, and/or a stabilizer(s), and/or a preservative (s) and/or an isotonicity agent(s).
233. A method of reducing the blood glucose level in mammals by administering to a patient in need of such treatment a therapeutically active dose of an insulin preparation according to any one embodiments 219-230.
234. A method for the treatment of diabetes mellitus in a subject comprising administering to a subject an insulin preparation according to any one of embodiments 219-231.
235. An insulin preparation according to any one of embodiments 219-232, for use in the treatment or prevention of hyperglycemia including stress induced hyperglycemia, type 2 diabetes, impaired glucose tolerance, type 1 diabetes, and burns, operation wounds and other diseases or injuries where an anabolic effect is needed in the treatment, myocardial infarction, stroke, coronary heart disease and other cardiovascular disorders and treatment of critically ill diabetic and non-diabetic patients.

The invention is further illustrated by the following examples which are not to be construed as limiting.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### Example 1

### Preparation of Pharmaceutical Preparations

The pharmaceutical preparations of the present invention may be formulated as an intermediate preparation of a long-acting insulin compound added phenol and or a phenolic preservative followed by zinc acetate or zinc chloride combined with an intermediate preparation of a fast acting insulin compound. The combined preparation must include nicotinamide and arginine, which may be added to one or both intermediate preparations or separately. The combined preparation may further include a buffer, e.g. trishydroxymethylaminomethane (tris), and is made isotonic with e.g. glycerol.

### Manufacturing of Preparations A-H

Insulin degludec stock solution was made by suspending 2566 mg insulin compound (152 nmol/mg) in 120 mL water for injection (water) and 0.2 N sodium hydroxide added to pH 7.4 followed by water for injection to 130.5 g corresponding to 3000 μM. The solution was filtered through a 0.22 μm sterile filter.

Excipient stock solutions were prepared including adjustment of pH to about 7.4 of glycerol to 2100 mmol/kg, of phenol to 320 mmol/kg, of m-cresol to 160 mmol/kg, of arginine hydrochloride to 500 mmol/kg, of sodium chloride to 500 mmol/kg, and of nicotinamide to 2400 mmol/kg. Finally a solution of 10 mM zinc acetate was made.

Excipient stock solutions except nicotinamide and zinc acetate were combined, eg. for preparation D: 3.91 g glycerol solution, 2.16 g phenol solution (a factor of 1.03 was employed to correct for preservative loss during manufacturing), 4.32 g m-cresol solution, 1.2 g arginine hydrochloride solution, and 10 g water followed by addition of 8.4 g degludec 3000 μM. Zinc acetate solution was added in portions of 1 Zn/6ins per 2 minutes to 4.7 Zn/6degludec, pH adjusted to 7.4 by 0.2N sodium hydroxide and water to 40.2 g. The solution was stored over night before combination with 18.1 g 600 μM insulin aspart formulation including preservatives and zinc and finally 2.0 g 2400 mmol/kg nicotinamide stock solution. Finally the combined formulation was filtered through a sterile filter and transferred to carpoules for injection systems.

Insulin aspart intermediate preparation was made by suspending 1192 mg aspart (151 nmol/mg) in 60 g water and addition of 5.4 equivalents of hydrochloric acid, 0.5 equivalents of zinc acetate and water ad 150 g, followed by addition of 30.9 g m-cresol stock solution and 15.45 g phenol stock solution, adjustment of pH to 7.4 by 0.2 N sodium hydroxide and water ad 300.9 g.

TABLE 1
Composition of insulin preparations according to this invention

| | Insulin | Phenol/ | Zn/6ins | Insulin | Zn/6ins | Nicotin- | Arginine, | | NaCl or | Zn | |
|---|---|---|---|---|---|---|---|---|---|---|---|
| | degludec | m-cresol | long- | aspart | fast- | amide | HCl | Glycerol | Tris | final | |
| | (μM) | (mM) | acting | (μM) | acting | (mM) | (mM) | (mM) | (mM) | μM | pH |
| A | 420 | 16/16 | 4.7 | 180 | 3.00 | 0 | 0 | 210 | NaCl 10 | 419 | 7.4 |
| B | 420 | 16/16 | 4.7 | 180 | 3.00 | 230 | 10 | 0 | 0 | 419 | 7.4 |
| C | 420 | 16/16 | 4.7 | 180 | 3.00 | 120 | 10 | 100 | 0 | 419 | 7.4 |
| D | 420 | 16/16 | 4.7 | 180 | 3.00 | 80 | 10 | 137 | 0 | 419 | 7.4 |
| E | 420 | 16/16 | 4.7 | 180 | 3.00 | 40 | 10 | 173 | 0 | 419 | 7.4 |
| F | 420 | 16/16 | 4.70 | 180 | 3.00 | 0 | 10 | 210 | 0 | 419 | 7.4 |
| G | 420 | 16/16 | 4.70 | 180 | 3.00 | 40 | 10 | 160 | Tris 7 | 419 | 7.4 |
| H | 420 | 16/16 | 4.70 | 180 | 3.00 | 80 | 10 | 124 | Tris 7 | 419 | 7.4 |

### Manufacturing of Preparations I-J

### Preparation I

Insulin detemir stock solution is made by dissolving 2114 mg insulin compound (141.9 nmol detemir/mg, 2.3 Zn/6ins, 1 phenol/ins) in 55 g water, adjusting to pH 7.4. and water to 75 g corresponding to 4000 μM. The solution is filtered through a 0.22 μm sterile filter.

Excipient stock solutions are prepared including adjustment of pH to about 7.4 of glycerol to 2100 mmol/kg, of phenol to 320 mmol/kg, of m-cresol to 160 mmol/kg, of arginine hydrochloride to 500 mmol/kg, of tris to 500 mmol/kg, and of nicotinamide to 2400 mmol/kg. Finally a solution of 10 mM zinc acetate is made.

Intermediate detemir preparation is made by combining excipient stock solutions except nicotinamide and zinc acetate before addition of insulin detemir: 3.53 g glycerol solution, 2.25 g phenol solution, 5.14 g m-cresol solution, 1.2 g arginine hydrochloride solution, 0.84 g tris solution and 25.2 g detemir stock solution (4000 μM). 359 μL zinc acetate solution (9.37 μM) is added ad 2.5 Zn/6detemir, pH adjusted to 7.4 by 0.2N sodium hydroxide and water to 42.2 g.

Insulin aspart intermediate preparation is made by suspending 1192 mg aspart (151 nmol/mg) in 60 g water and adding of 5.4 equivalents of hydrochloric acid, 0.5 equivalents of zinc acetate and water ad 150 g, followed by addition of 30.9 g m-cresol stock solution and 15.45 g phenol stock solution, adjustment of pH to 7.4 by 0.2 N sodium hydroxide and water ad 300.9 g.

The intermediate detemir preparation is stored over night before combination with 18.1 g 600 μM intermediate aspart preparation including preservatives and zinc. Finally the combined formulation is filtered through a sterile filter and transferred to carpoules for injection systems.

### Preparation J

Preparation J is manufactured like preparation I except reducing total weight adjustment by water to detemir intermediate preparation by 2.0 g and adding 2.0 g 2400 mmol/kg nicotinamide stock solution to the combined detemir and aspart preparation.

TABLE 2
Composition of insulin preparations according to this invention

| | Insulin | Phenol/ | Zn/6ins | Insulin | Zn/6ins | Nicotin- | Arginine, | | NaCl or | Zn | |
|---|---|---|---|---|---|---|---|---|---|---|---|
| | detemir | m-cresol | long- | aspart | fast- | amide | HCl | Glycerol | Tris | final | |
| | (μM) | (mM) | acting | (μM) | acting | (mM) | (mM) | (mM) | (mM) | μM | pH |
| I | 1680 | 19/19 | 2.5 | 180 | 3.00 | 0 | 10 | 124 | 7 | 790 | 7.4 |
| J | 1680 | 19/19 | 2.5 | 180 | 3.00 | 80 | 10 | 124 | 7 | 790 | 7.4 |

### Manufacturing of Preparations 1-7

Insulin degludec intermediate preparation (all concentrations multiplied by 7/6) was made by suspending 834 mg insulin compound (151 nmol/mg) in 100 mL water and 0.2 N sodium hydroxide added to pH 7.8, followed by 21.6 g phenol solution (320 mmol/kg), 10.53 g zinc acetate solution (9.37 mM), 21.0 g nicotinamide solution (2600 mmol/kg) and water to 180.5 g after adjusting pH to 7.4. The solution was filtered through a 0.22 μm sterile filter.

Insulin aspart intermediate preparation was made by suspending 397 mg aspart (151 nmol/mg) in 20 g water and addition of 324 μL 1 N of hydrochloric acid, 3.20 g of zinc acetate solution (9.37 mM) and water ad 50 g, followed by addition of 10.3 g phenol stock solution (320 mmol/kg), adjustment of pH to 7.4 by 0.2 N sodium hydroxide, addition of 10.0 g nicotinamide solution (2600 mmol/kg) and water ad 100.3 g. The solution was filtered through a 0.22 μm sterile filter.

Stock solutions of pH 7.4 of arginine hydrochloride, glutamic acid, and glycine were made to 500 mmol/kg and histidine to 300 mmol/kg using sodium hydroxide/hydrochloric acid for pH adjustment. The stock solutions were filtered through a 0.22 μm sterile filter.

The final preparations shown in Table 3 were made by 18.0 g insulin degludec intermediate preparation added amino acid stock solution+eventually water to 3.0 g and finally combining with 9.0 g aspart intermediate preparation. The preparations were transferred to carpoules for injection systems and stored 2 weeks at 37 C or 5 C for determination of physical and chemical stability.

TABLE 3
Composition of further insulin preparations according to this invention

| | Insulin | | | Insulin | | Nicotin- | | | | | Zn | |
|---|---|---|---|---|---|---|---|---|---|---|---|---|
| | degludec | Phenol | Zn/6ins | aspart | Zn/6ins | amide | Arg | Glu | His | Gly | final | pH |
| | (μM) | (mM) | degludec | (μM) | aspart | (mM) | mM | mM | mM | mM | (μM) | final |
| 1 | 420 | 32 | 4.70 | 180 | 3.00 | 260 | 0 | 0 | 0 | 0 | 419 | 7.4 |
| 2 | 420 | 32 | 4.70 | 180 | 3.00 | 260 | 10 | 0 | 0 | 0 | 419 | 7.4 |
| 3 | 420 | 32 | 4.70 | 180 | 3.00 | 260 | 30 | 0 | 0 | 0 | 419 | 7.4 |
| 4 | 420 | 32 | 4.70 | 180 | 3.00 | 260 | 50 | 0 | 0 | 0 | 419 | 7.4 |
| 5 | 420 | 32 | 4.70 | 180 | 3.00 | 260 | 0 | 50 | 0 | 0 | 419 | 7.4 |
| 6 | 420 | 32 | 4.70 | 180 | 3.00 | 260 | 0 | 0 | 30 | 0 | 419 | 7.4 |
| 7 | 420 | 32 | 4.70 | 180 | 3.00 | 260 | 0 | 0 | 0 | 50 | 419 | 7.4 |

### Example 2

### Analysis of Insulin Chemical Stability

### Size Exclusion Chromatography

Quantitative determination of high molecular weight protein (HMWP) and monomer insulin aspart was performed on Waters insulin (300×7.8 mm, part nr wat 201549) with an eluent containing 2.5 M acetic acid, 4 mM L-arginine and 20% (V/V) acetonitrile at a flow rate of 1 ml/min. and ambient temperature. Detection was performed with a tunable absorbance detector (Waters 486) at 276 nm. Injection volume was 40 μl and a 600 μM human insulin standard. HMWP and concentration of the preparations were measured at each sampling point.

### Reverse Phase Chromatography

Determination of the insulin aspart related impurities were performed on a HPLC system using a RP C18, 4.6×150 mm column, particle size of 3.5 μm Waters Sunfire with a flow rate of 1 ml/min., at 43° C. and detection at 214 nm. Elution was performed with a mobile phase consisting of the following:

A. 7.7% (w/w) acetonititrile, 2.8% (w/w) sodium sulphate, 0.27% (w/w) o-phosphoric acid, pH 3.6.

B. 42.8% (w/w) acetonitrile. Gradient: 0-20 min isocratic with 58%/42% of A/B (aspart main peak adjusted to about 16 min), 20-32 min linear change to 10%/90% A/B, 32-33 min. linear change to initial condition and run time of 40 min.

The amount of B28 iso-aspartate, desamido and other related impurities were determined as absorbance area measured in percent of total absorbance area determined after elution of the preservatives until 28 min. Insulin degludec was eluting about 30 min.

Determination of insulin degludec related impurities were performed on a HPLC system using a RP C8, 4.6×150 mm column, particle size of 3.5 μm Waters Symmetry Shield with a flow rate of 1 ml/min., at 40° C. and detection at 214 nm. Elution was performed with a mobile phase consisting of the following:

A. 7.7% (w/w) acetonititrile, 1.42% (w/w) sodium sulphate, 1.38% (w/w) sodium dihydrogenphosphate monohydrate adjusted to pH 5.9 by sodium hydroxide.

B. 42.8% (w/w) acetonitrile. Gradient: 0-45 min isocratic with 50%/50% of A/B (degludec main peak adjusted to about 20 min), 45-50 min linear change to 20%/80% A/B, 51 min sudden change to initial condition, and run time of 60 min.

The amount of hydrophilic impurities of insulin degludec was determined as absorbance area in percent of total absorbance area after elution of m-cresol about 10 min until the main peak, hydrophobic impurities 1 from the main peak to start of the gradient, and hydrophobic impurities 2 as the area of peaks eluted by the gradient.

The preparations shown in Table 3 were analyzed for chemical impurities of insulin aspart and insulin degludec determined as the difference between preparations stored in carpoules in 2 weeks at 37 C and at 5 C. The results are shown in Table 4.

TABLE 4
Physical and chemical stability data for insulin preparations of Table 3 (Example 1)

| | Physical | Chemical stability, aspart | | | Chemical stability, degludec | | | Chemical sta-, |
|---|---|---|---|---|---|---|---|---|
| | stability | Δ % degrad/2 w 37 C. | | | Δ % degrad/2 w 37 C. | | | bility aspart + |
| | lag time | | Des- | Other | Hydro- | Hydro- | Hydro- | degludec |
| Prep. | (min), | B28 | amido | related | phil. | phob. | phob. | Δ % HMWP/ |
| no | ThT-assay | isoAsp | forms | imp. | Imp. | imp. 1 | imp. 2 | 2 w 37 C. |
| 1 | 100 | 1.34 | 3.48 | 0.19 | 0.17 | 1.02 | 0.29 | 0.19 |
| 2 | 100 | 1.33 | 2.83 | 0.11 | 0.14 | 0.67 | 0.13 | 0.11 |
| 3 | 60 | 1.35 | 2.96 | 0.10 | 0.31 | 0.49 | 0.12 | 0.10 |
| 4 | 60 | 1.37 | 2.30 | 0.02 | 0.41 | 0.38 | 0.16 | 0.02 |
| 5 | 60 | 1.45 | 3.24 | 0.12 | 0.35 | 0.60 | 0.27 | 0.12 |
| 6 | 20 | 1.52 | 3.92 | 0.10 | 3.45 | 0.52 | 0.55 | 0.10 |
| 7 | 60 | 1.40 | 3.54 | 0.13 | 0.39 | 0.73 | 0.57 | 0.13 |

Addition of arginine reduces the amount of degradation products formed, especially HMWP and des-amido forms. Increasing the concentration of arginine in the range 10 to 50 mM leads to further reduction of degradation.

The physical stability measured as lag time in the ThT assay is reduced upon addition of 30 mM and 50 mM arginine and unchanged at 10 mM arginine.

The overall performance of 50 mM arginine is superior to 50 mM glycine or 50 mM glutamic acid, and 30 mM arginine is superior to 30 mM histidine regarding reduction of the formation of degradation products, as shown in Table 3.

### Example 3

### Pharmacokinetic (PK)/Pharmacodynamic (PD) Studies in LYD Pig Model and Plasma Analysis Assay

### PK/PD Studies in LYD Pigs

The PK/PD studies were performed on domestic female pigs, LYD cross-breed, weighing between 55 and 110 kg. The pigs were catheterised into the jugular vein through an ear vein at least 2 days before start of the study. The last meal before the start of the study was served to the animals approx. 18 hours prior to the injection of the test preparation, and the animals had free access to water at all time during the fasting period and the test period.

At time 0 hours the test preparation was given subcutaneous on the lateral side of the neck. A blood sample was drawn prior dosing and at regular time intervals after dosing samples were drawn from the catheter and sampled into 1.5 ml glass tubes pre-coated with heparin. The blood samples were kept in ice water until separation of plasma by centrifugation for 10 min. 3000 rpm at 4° C., which was done within the first 30 minutes. Plasma samples were stored at 4° C. for short time (2-3 hours) or at −18° C. for long term storage and were analysed for glucose on YSI or Konelab 30i and for insulin Aspart concentration by LOCI.

### Luminescent Oxygen Channeling Immunoassay (LOCI) for Insulin Aspart and Insulin Degludec Quantification

The insulin aspart and insulin degludec LOCI are monoclonal antibody-based sandwich immunoassays and applies the proximity of two beads, the europium-coated acceptor beads and the streptavidin coated donor-beads. The acceptor beads were coated with a specific antibody against human insulin and recognize insulin Aspart in plasma samples. A second biotinylated antibody bind specific to insulin Aspart and together with the streptavidin coated beads, they make up the sandwich. Illumination of the beads-aggregate-immunocomplex releases singlet oxygen from the donor beads which channels into the acceptor beads and triggers chemiluminescence. The chemiluminescence was measured and the amount of light generated is proportional to the concentration of insulin aspart. Likewise a specific LOCI assay for insulin degludec was used.

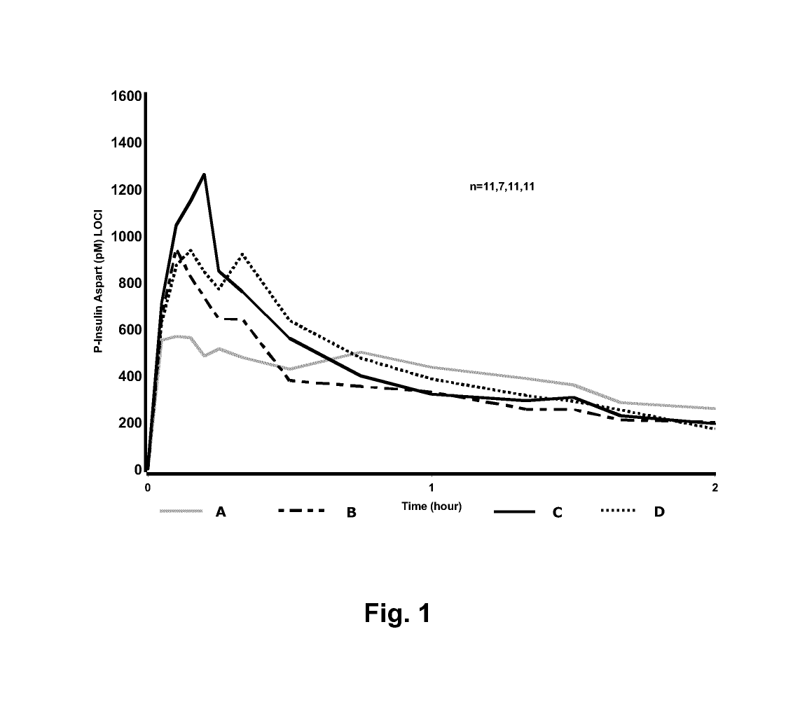

Compared to the product in Phase III, Boost™ (preparation A, without nicotinamide), the initial absorption rate of insulin aspart is faster for the preparations comprising 230 mM, 120 mM, or 80 mM nicotinamide (preparations B, C and D) included in the present invention (FIG. 1).

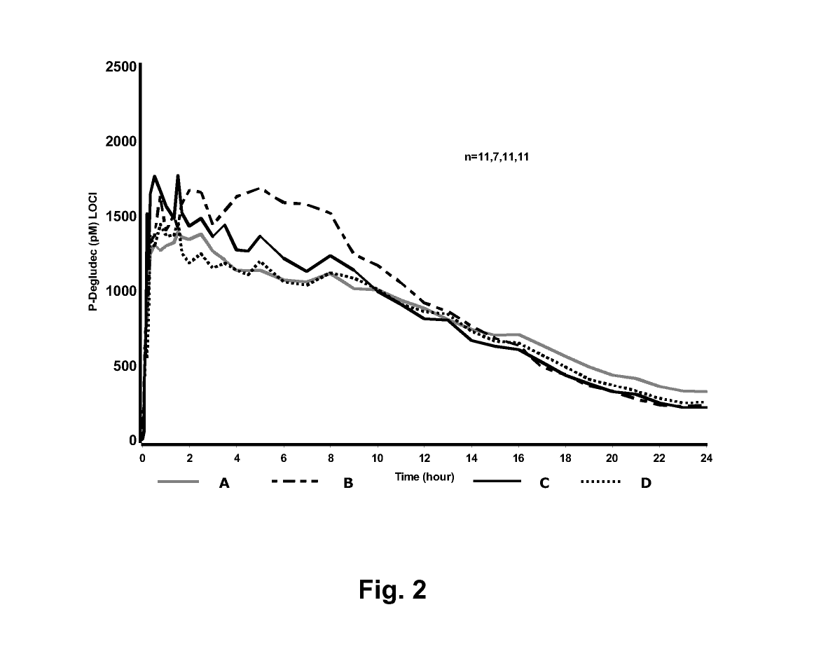

In the same pig experiments the absorption profile of insulin degludec is measured. At high nicotinamide concentration of 230 mM the kinetic profile was changed to intermediary high plasma concentrations of degludec whereas the degludec profile was not affected at 120 mM and 80 mM nicotinamide (FIG. 2).

### Example 4

### General Introduction to ThT Fibrillation Assays for the Assessment of Physical Stability of Protein Formulations

Low physical stability of a peptide may lead to amyloid fibril formation, which is observed as well-ordered, thread-like macromolar structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284]. The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t₀ is the time needed to reach 50% of maximum fluorescence. The two important parameters describing fibril formation are the lag-time calculated by t₀−2τ and the apparent rate constant kₐₚₚ=1/τ.

Time Course for Fibril Formation:

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

### Sample Preparation

Samples were prepared freshly before each assay. Each sample composition is described in each example. The pH of the sample was adjusted to the desired value using appropriate amounts of concentrated NaOH and HClO₄ or HCl. Thioflavin T was added to the samples from a stock solution in H₂O to a final concentration of 1 μM.

Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

### Incubation and Fluorescence Measurement

Incubation at given temperature, shaking and measurement of the ThT fluorescence emission were done in a Fluoroskan Ascent FL fluorescence platereader or Varioskan platereader (Thermo Labsystems). The temperature was adjusted to 37° C. The orbital shaking was adjusted to 960 rpm with an amplitude of 1 mm in all the presented data. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

### Data Handling

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, since full sigmodial curves are not always achieved during the measurement time, lag times were here visually determined from the ThT fluorescence curve as the time point at which the ThT fluorescence is different than the background level.

### Measurement of Initial and Final Concentrations

The peptide concentration in each of the tested formulations were measured both before application in the ThT fibrillation assay (“Initial”) and after completion of the ThT fibrillation (“After ThT assay”). Concentrations were determined by reverse HPLC methods using a pramlin-tide standard as a reference. Before measurement after completion 150 μl was collected from each of the replica and transferred to an Eppendorf tube. These were centrifuged at 30000 G for 40 mins. The supernatants were filtered through a 0.22 μm filter before application on the HPLC system.

### Example 5

### In Vitro Model

Size exclusion chromatography has been used as an in vitro model for insulin detemir disappearance from a subcutaneous depot into the blood compartment [ref. Pharmaceutical Research, 21 (2004) 1498-1504] as well as for insulin compounds self-associating to a high molar mass complex after removal of the preservatives phenol and m-cresol from the pharmaceutical preparation [Pharmaceutical Research, 23 (2006) 46-55]. Evaluation of mixtures or combinations of a long acting and a fast acting insulin compound by size exclusion chromatography is described in PCT WO 2007/074133, and for a reference preparation of insulin degludec and insulin aspart according to this invention insulin degludec elutes at the exclusion limit of a superpose 6 column about a size of a high molar mass complex larger than a 5 mega dalton protein. This high molar mass complex formed after removal of phenol is anticipated to be main factor in protraction of multihexamer-forming insulin compounds. The fast acting insulin compound elutes in the insulin monomer region at the end of the chromatogram.

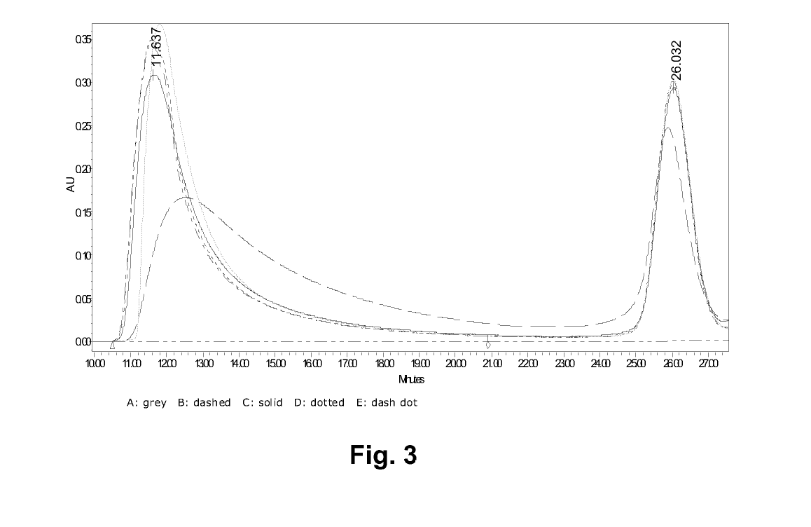

The SEC in vitro model has been employed on preparations included in table 1 and examples are shown in FIG. 3. Multihexamer formation of insulin degludec in preparations combined with insulin aspart according to table 1 was reduced by including 230 mM (dashed line) or 120 mM nicotinamide (solid line) whereas the peak height of the multihexameric complex was about the same for preparations including 80 mM (dotted line) or 40 mM nicotinamide (dash dot line) as a reference preparation without nicotinamide (grey solid).

Method: Column: Superose 6PC (0.32*30 cm). Eluent: saline buffered with 10 mM tris and 37 C. Flow: 80 μL/min. Injection volume: 20 μL and UV detection at 276 nm.

### Example 6

### Method for Determination of Dihexamer Insulin Content in a Pharmaceutical Preparation

Size exclusion chromatography was performed on a Waters BEH200 SEC column at ambient temperature using 140 mM sodium chloride, 2 mM phenol and 10 mM tris pH 7.3 at a flow rate of 300 μL/min. 20 μL samples were injected and UV detection at 276 nm. References were a monomer insulin (Asp^{B9}, GIu^{B27}, human insulin; RT=5.9 min), Co(III)insulin-hexamer (hexamer RT=4.9 min; dihexamer RT=4.4 min), and human albumin (albumin RT=4.2 min; albumin dimer RT=3.7 min). The area were divided in peaks referring to tetrahexamer and higher molar mass associates of insulin, dihexamer insulin, hexamer insulin, and monomer and dimer insulin.

1. An insulin preparation comprising:
an acylated insulin or an analog thereof,
human insulin or an analog thereof,
a nicotinic compound, and
arginine.

2. The insulin preparation according to claim 1, wherein the acylated insulin or analog thereof is an insulin acylated in the ε-amino group of a Lys residue in a position in the B-chain of the parent insulin molecule.

3. The insulin preparation according to claim 2, wherein the parent insulin is selected from the group consisting of human insulin; desB1 human insulin; desB30 human insulin; GlyA21 human insulin; GlyA21 desB30 human insulin; AspB28 human insulin; porcine insulin; LysB28 ProB29 human insulin; LysB3 GluB29 human insulin and AspB28 desB30 human insulin.

4. The insulin preparation according to claim 2, wherein the acylated insulin is NεB29-hexadecandiyol-γ-Glu-(desB30) human insulin.

5. The insulin preparation according to claim 2, wherein the acylated insulin is N^{εB29}-myristoyl (desB30) human insulin.

6. The insulin preparation according to claim 1, wherein the insulin analog is B28Asp human insulin.

7. The insulin preparation according to claim 1, wherein the insulin analog is selected from the group consisting of B28LysB29Pro human insulin and B3LysB29Glu human insulin.

8. The insulin preparation according to claim 1, wherein the human insulin or analog thereof and acylated insulin or analog thereof are present in the amount from about 0.1 mM to about 10.0 mM.

9. The insulin preparation according to claim 1, wherein the acylated insulin or analog thereof is present in about 70% and the human insulin or insulin analog is present in about 30%.

10. The insulin preparation according to claim 1, wherein the nicotinic compound is selected from the group consisting of nicotinamide, nicotinic acid, niacin, niacin amide and vitamin B3 and/or salts thereof and/or any combination thereof.

11. The insulin preparation according to claim 1, wherein the nicotinic compound is nicotinamide.

12. The insulin preparation according to claim 1, comprising from about 1 mM to about 120 mM of arginine.

13. The insulin preparation according to claim 1, further comprising one or more of a buffer, a metal ion, a stabilizer, a preservative or an isotonicity agent.

14. A method of reducing the blood glucose level in mammals by administering to a patient in need of such treatment a therapeutically active dose of an insulin preparation according to claim 1.

15. A method for the treatment of diabetes mellitus in a subject comprising administering to a subject an insulin preparation according to claim 1.

16. (canceled)

17. The insulin preparation according to claim 4, wherein the insulin analog is B28Asp human insulin.
